(19) United States
(12) Patent Application Publication
(10) Pub. No.: US 2026/0098858 A1
(43) Pub. Date: Apr. 9, 2026

(54) Fc-Engineered Monoclonal Antibodies Refractory to Tumor Immunosuppressive ICAM-1/CD54

(71) Applicant: NAVROGEN, INC., Glen Mills, PA (US)

(72) Inventors: James Bradford Kline, Morgantown, PA (US); Nicholas C. Nicolaides, Glen Mills, PA (US); Luigi Grasso, Bryn Mawr, PA (US)

(73) Assignee: NAVROGEN, INC., Glen Mills, PA (US)

(21) Appl. No.: 19/104,036

(22) PCT Filed: Aug. 14, 2023
(86) PCT No.: PCT/US23/72162

§ 371 Date: Feb. 14, 2025

Related U.S. Application Data
(60) Provisional application No. 63/371,432, filed on Aug. 15, 2022.

(51) Int. Cl. G01N 33/50; C07K 16/28; C40B 30/06; G01N 33/68

(52) U.S. Cl. CPC G01N 33/5011; C07K 16/2821; C40B 30/06; G01N 33/6845; C07K 2317/732

(57) ABSTRACT

The ICAM-1/CD54 protein has been found to be a suppressor of antibody-mediated humoral immunity facilitated through its direct binding to the CH3 domain within IgG1 heavy chains. Its binding suppresses antibody immune-effector activity by reducing IgG1 engagement with Fc receptors on effector cells and C1q complement initiating protein. In addition, its binding has the potential to reduce antibody drug conjugate activity by slowing target cell internalization. Engineered antibodies with CH3 region modifications can be generated that reduce or eliminate ICAM-1/CD54 binding leading to antibodies with improved immune-effector activity and antibody drug conjugate killing in the presence ICAM-1/CD54. These antibodies can be useful to treat patients with cancer and inflammatory and infectious diseases exhibiting elevated ICAM-1/CD54 levels.

### TECHNICAL FIELD OF THE INVENTION

This invention relates to the area of humoral immunity and humoral immuno-oncology. In particular, it relates to methods and compositions of agents that can overcome the immunosuppressive effects of the ICAM-1/CD54 protein to improve antibody-based therapeutic efficacy in inhibiting cancer growth and other humoral immunosuppressed diseases.

### REFERENCE TO AN ELECTRONIC SEQUENCE LISTING

The contents of the electronic sequence listing (008966.00037 sequence listing.xml; Size: 15 kilobytes; and Date of Creation: Jul. 31, 2023) is herein incorporated by reference in its entirety.

### BACKGROUND OF THE INVENTION

Humoral immunity is a major mechanism by which vertebrate host organisms surveil and defend against dysregulated and transformed host cells. In cancer biology, immune checkpoint inhibitors that can overcome suppressed cellular-mediated immunity have demonstrated robust effects in unleashing activated CD8+ T-cell killing against subsets of tumors (Hodi F S, et al. N Engl J Med 363:711-723, 2010). Several commercially approved therapeutic antibodies have been reported to exhibit their tumor-killing effects through humoral-mediated antibody-dependent cellular cytotoxicity (ADCC), antibody-dependent cellular phagocytosis (ADCP) and complement-dependent cytotoxicity (CDC) (DiLillo D J, Ravetech J V, Cancer Immunol Res 3:704-713, 2015; Ruck T, et al. Int J Mol Sci. 16:16414-16439, 2015; Pelaia C, et al. Biomed Res Int 4839230:1-9, 2018). Recent translational findings have shown that tumors produce factors that can suppress humoral immune pathways and in turn suppress the tumor-killing effects via ADCC, ADCP and CDC (Vergote I, et al. J Clin Oncol 34:2271-2278; Kline J B, et al. J Clin Oncol 5:15, 2018; Wang W et al. Cytogenet Genome Res 152:169-179, 2017; Kline J B et al. Eur J Immunol. 48:1872-1882, 2018; Grasso L et al. Oncol Letters 23:2, 2022). Moreover, these tumor-produced factors have also been shown to bind to antibody components within antibody drug conjugates (ADCs) that reduce their internalization and overall target cell killing (Nicolaides N C, et al. PloS ONE DOI.org/10.1371/journal.pone.0285161, 2023).

The antibody-mediated humoral immune response is governed by the coordination of antibody-cell surface antigen engagement that positions the antibody on the antigen epitope at a certain proximity to the cell surface. Once bound, antibodies may engage with Fc-γ activating receptors FCGR3A (CD16a) and FCGR2A (CD32a) on Natural Killer (NK) or dendritic/myeloid/monocytic cells, respectively, (any cell that participates in ADCC is referred to herein as an “immune-effector cell”) to initiate ADCC or antibody-dependent cellular phagocytosis (ADCP) as well as engage with the C1q complement initiating protein to elicit target cell death of antibody-bound cells via the classical complement CDC pathway (Reuschenbach M, et al. Cancer Immunol Immunother 58:1535-1544, 2009). These effects have been observed for several therapeutic antibodies, such as but not limited to rituximab, trastuzumab, cetuximab, pertuzumab, daratumumab, and alemtuzumab (Zhou X, et al. Oncologist 13:954-966, 2008; Hsu Y F, et al. Mol Cancer 9:1-8, 2010; Spiridon C I, et al. Clin Cancer Res 8:1720-1730, 2002; Luo C, et al. Sci Rep 7:46347, 2017; Casneuf T et al. Blood Adv 1:2105-2114, 2017).

Several reports have found that the tumor-produced soluble and membrane-bound forms of ICAM-1 are associated with poor prognosis in patients with gastric cancer, NSCLC, melanoma, breast cancer, colorectal cancer, multiple myeloma and lymphoma (Maruo Y et al. Int J Cancer 100:486-490, 2002; Wu M et al. Path Res Pract 10.1016/j.prp.2020.153029; Roland C L et al. Surgery 141:705-707, 2007).). Several of these cancer indications are treated with antibody-based therapies that employ immune-effector mechanisms for tumor killing.

There is a continuing need in the art to develop tools and agents that may overcome the humoral immunosuppression and/or reduced efficacy of ADCs mediated by the soluble and membrane-bound form of ICAM-1/CD54 (referred to herein as ICAM-1) in cancer patients as well as other diseases in which humoral immunosuppression associated with ICAM-1 occurs.

### SUMMARY OF THE INVENTION

One aspect of the invention is an immunosuppression-refractory human IgG1 antibody with from one to four amino acid substitutions relative to an immunosuppression-susceptible human IgG1 antibody without the one to four amino acid substitutions. The immunosuppression of the immunosuppression-susceptible human IgG1 is caused by soluble or membrane-bound ICAM-1/CD54.

Another aspect of the invention is a polynucleotide encoding an immunosuppression-refractory human IgG1 antibody. The immunosuppression-refractory human IgG1 antibody has from one to four amino acid substitutions relative to an immunosuppression-susceptible human IgG1 antibody without the one to four amino acid substitutions. The immunosuppression of the immunosuppression-susceptible human IgG1 is caused by soluble or membrane bound ICAM-1/CD54.

Another aspect of the invention is a nucleic acid vector encoding an immunosuppression-refractory human IgG1 antibody. The immunosuppression-refractory human IgG1 antibody has from one to four amino acid substitutions relative to an immunosuppression-susceptible human IgG1 antibody without the one to four amino acid substitutions. The immunosuppression of the immunosuppression-susceptible human IgG1 is caused by soluble or membrane bound ICAM-1/CD54.

Another aspect of the invention is a stable cell line comprising a nucleic acid vector. The stable cell line expresses an immunosuppression-refractory human IgG1 antibody. The immunosuppression-refractory human IgG1 antibody has from one to four amino acid substitutions relative to an immunosuppression-susceptible human IgG1 antibody without the one to four amino acid substitutions. The immunosuppression of the immunosuppression-susceptible human IgG1 is caused by soluble or membrane bound ICAM-1/CD54.

Yet another aspect of the invention is a method of treating a patient with a disease wherein the patient expresses an elevated level of ICAM-1/CD54 compared to a population of healthy humans. An immunosuppression-refractory human IgG1 antibody in a canonical or antibody drug conjugate (ADC) format is administered to the patient. The immunosuppression-refractory human IgG1 antibody has from one to four amino acid substitutions relative to an immunosuppression-susceptible human IgG1 antibody without the one to four amino acid substitutions. The immunosuppression of the immunosuppression-susceptible human IgG1 is caused by soluble or membrane-bound ICAM-1/CD54.

Another aspect of the invention is a method of treating a cancer patient or a patient with an inflammatory disease. A full-length human IgG1 antibody is administered to the cancer patient or the patient with an inflammatory disease. The antibody comprises a heavy chain with one to four amino acid substitutions in motifs within Kabat region 367-425, preferably in Kabat region 369-410, which is located within a heavy chain of the full-length human IgG1 antibody. In one embodiment the amino acid substitutions occur in amino acid residues YSKL (407-410) (SEQ ID NO: 3).

Still another aspect of the invention is a method of screening candidate antibodies to identify those that are refractory to ICAM-1/CD54 immunosuppression. (a) A human cancer cell line expressing an antigen is contacted with a candidate IgG1-type antibody which specifically binds to the antigen. The contacting is performed in the presence of ICAM-1/CD54 (SEQ ID NO: 1). (b) A human cancer cell line expressing the antigen is contacted with the candidate IgG1-type antibody which specifically binds to the antigen. The contacting is performed in the absence of ICAM-1/CD54 (SEQ ID NO: 1). (c) Antibody dependent cellular cytotoxicity (ADCC) or complement dependent cytotoxicity (CDC) of the human cancer cell line stimulated by the candidate antibody in steps (a) and (b) is determined. (d) The antibody may be formatted as an antibody drug conjugate (ADC). Target cell killing is determined by comparing that achieved in step (a) to step (b).

Another aspect is a method of screening a candidate immunosuppression-refractory human IgG1 antibody with from one to four amino acid substitutions relative to an immunosuppression-susceptible human IgG1 antibody to identify a candidate that is refractory to ICAM-1/CD54 immunosuppression. The candidate immunosuppression-refractory human IgG1 antibody is contacted with biotinylated human CD16a Fc receptor. The contacting is performed in the presence of sICAM-1/CD54 as shown in SEQ ID NO: 2. The immunosuppression-susceptible human IgG1 antibody is contacted with biotinylated human CD16a Fc receptor. The contacting is performed in the presence of sICAM-1/CD54 as shown in SEQ ID NO: 2. The binding of CD16a Fc receptor by the candidate immunosuppression-refractory and the immunosuppression-susceptible IgG1 antibodies is determined.

Another aspect of the invention is a method of screening a candidate immunosuppression-refractory human IgG1 antibody with from one to four amino acid substitutions relative to an immunosuppression-susceptible human IgG1 antibody to identify a candidate that is refractory to ICAM-1/CD54 immunosuppression. The candidate immunosuppression-refractory human IgG1 antibody is contacted with biotinylated human C1q protein. The contacting is performed in the presence of SICAM-1/CD54 as shown in SEQ ID NO: 2. The immunosuppression-susceptible human IgG1 antibody is contacted with biotinylated human C1q protein. The contacting is performed in the presence of sICAM-1/CD54 as shown in SEQ ID NO: 2. The binding of biotinylated human C1q protein receptor by the candidate immunosuppression-refractory human IgG1 antibody and the immunosuppression-susceptible human IgG1 antibody is determined.

Another aspect of the invention is a method of screening a candidate immunosuppression-refractory human IgG1 antibody in an antibody drug conjugate (ADC) format with from one to four amino acid substitutions relative to an immunosuppression-susceptible human IgG1 antibody to identify a candidate that is refractory to ICAM-1/CD54 immunosuppression. The candidate immunosuppression-refractory ADC is incubated with isogenic ICAM-1 expressing and non-ICAM-1 expressing cells and the comparative killing of each cell type is determined.

Yet another aspect of the invention is a method to identify tumor-bearing patients who are good candidates for treatment with an antibody-drug conjugate. A tumor from each of a plurality of patients is tested for ICAM-1 expression. The testing determines whether the tumor from each of the plurality of patients expresses ICAM-1. If the tumor does not express ICAM-1, then treatment of the patient with an IgG1 antibody-drug conjugate is recommended. The IgG1 antibody of the antibody-drug conjugate is immunosuppression-susceptible. If the tumor expresses ICAM-1, then it is recommended that the patient not be treated with an IgG1 antibody-drug conjugate. Again, the IgG1 antibody of the antibody-drug conjugate is immunosuppression-susceptible. Optionally, at least one patient that does not express ICAM-1 is treated with an IgG1 antibody-drug conjugate. The IgG1 antibody of the antibody-drug conjugate is immunosuppression-susceptible.

These and other aspects of the invention, which will be apparent to those skilled in the art upon reading the specifications, provide the art with methods and compositions for use in improving antibody-mediated humoral immune responses and antibody drug conjugate efficacy in ICAM-1 immunosuppressed diseases, including cancer as well as non-oncologic diseases.

### BRIEF DESCRIPTION OF THE DRAWINGS

FIGS. 1A and 1B. Identification of ICAM-1 binding to human IgG1. Briefly, 96-well ELISA plates were coated with or without 1-10 μg/mL human soluble ICAM-1 (SICAM-1) or human serum albumin (HSA) and probed with 1-5 μg/mL rituximab, pertuzumab or trastuzumab as previously described. Kline J B, et al. OncoTarget 8:52045-52060, 2017). Each antibody was biotinylated and tested for binding to immobilized sICAM-1 via ELISA. Antibodies were biotinylated using EZ-Link™ Sulfo-NHS-Biotin (ThermoScientific) following the manufacturer's instructions. Biotinylated antibodies were quantified by Nanodrop and validated for signal intensity using an anti-IgG ELISA capture assay to measure the signal intensity for each antibody. All antibodies demonstrated similar signal intensity at similar concentrations (not shown). As shown in FIG. 1A, all antibodies were able to significantly bind to SICAM-1 similarly to trastuzumab (P=0.00009). To test for sICAM-1 binding to immobilized IgG1, 96-well ELISA plates were probed with 5 μg/mL of biotinylated SICAM-1 (Sino Biologicals) or biotinylated HSA both generated using the EZ-Link™ Sulfo-NHS-Biotin as above. As shown in FIG. 1B, SICAM-1 could significantly bind all antibodies similarly to pertuzumab (P=0.001), but not HSA. Experiments represent a minimum of triplicate wells. Statistical analysis was done using the Student's T-test.

FIGS. 2A-2B. SICAM-1 suppresses CD16a activation. To test for the ability of sICAM-1 to suppress CD16a Fc-γ-receptor activation and downstream ADCC, the Jurakt-CD16a-luciferase reporter system was utilized. In FIG. 2A, CD20-positive Daudi cells were used as target cells and the anti-CD20 rituxumab antibody was used for measuring CD16a activation in the presence of increasing amounts of sICAM-1 (0.1-10 μg/mL) following the manufacturer's protocol. As shown, cultures treated with sICAM-1 demonstrated suppressed CD16a activation in a dose-response manner with the highest dose having the most robust suppressive effect (P=0.0027). Experiments represent a minimum of triplicate wells. Statistical analysis were done using the Student's T-test. In similar fashion, a panel of other antibodies were analyzed for sICAM-1 inhibition of ADCC. As shown in FIG. 2B. 10 μg/mL sICAM-1 was capable of inhibiting CD16a activation of all antibodies dosed at 1 μg/mL.

FIGS. 3A and 3B. Inhibition of CD16a Fc receptor and C1q binding. Suppression of CD16a activation typically results in the reduced engagement of the CD16a Fc-Y-receptor with the antigen-bound IgG1 antibody. To determine if ICAM-1 binding to IgG1 reduces CD16a Fc-γ-receptor binding as well as the engagement of the complement mediating C1q protein, ELISA-based assays were performed to monitor their binding in the presence of sICAM-1. Briefly, pertuzumab was coated on 96-well plates and probed with biotinylated CD16a or C1q proteins in the presence of sICAM-1 (10 μg/mL). As shown, both CD16a (FIG. 3A) and C1q (FIG. 3B) protein binding was significantly reduced (P=0.00039 and 0.0033, respectively). Experiments represent a minimum of triplicate wells. Statistical analysis was done using the Student's T-test.

FIGS. 4A-4B. Domain mapping of ICAM-1 IgG1 binding. To localize the region where ICAM-1 potentially binds IgG1, human IgG1 fragments were generated through papain digestion to yield F(ab′)2 and Fc fragments. Whole IgG1 and fragments were immobilized on 96-well microplates (2.5 μg/mL) and probed with biotinylated sICAM-1 (0.5 μg/mL). As shown in FIG. 4A, SICAM-1 specifically binds to Fc domain (P=0.00081, as compared to HSA). Experiments represent a minimum of triplicate wells. Statistical analysis was done using the Student's T-test. Additionally, secreted recombinant monomeric heavy chain domains (CH2 and CH3) were expressed in 293F cells and tested in a similar manner (FIG. 4B). Interestingly, purified Fc and rituximab again exhibited binding of biotinylated sICAM-1, while none of the fragments bound. This further localized the ICAM-1 binding domain to the CH2-CH3 region.

FIGS. 5A-5C. IgG1 heavy chain deletion and mutagenesis. To further define the region where ICAM-1 binds IgG, GST-fusion proteins consisting of the Fc domain (Hinge region to the C-terminus, including a C-terminal FlagTag) were generated and used for competitive ICAM-1-IgG1 binding via ELISA. Deletion numbering was based on Kabat residue positions within IgG1 heavy chain as summarized in FIG. 5A. Constructs numbered 20-24 were also expressed as full-length variants of rituximab. As shown in FIG. 5A, GST deletion mutants from Kabat 411 to the C-terminus still significantly bound sICAM-1 (P=0.000144, constructs numbered 2 and 3). In contrast, Kabat 391 to 401 mutants (constructs numbered 4 and 5) lost ICAM-1 binding. FIG. 5B shows the results from a representative experiment using proteins with constructs numbered 3-5. To further define the region, multiple alanine and/or glycine substitution mutants were generated as both GST fusions as well as full-length rituximab antibodies for competitive binding of ICAM-1 to wild type IgG1 heavy chain (FIG. 5A, constructs numbered 6-24). The mutated heavy chain containing the alanine and/or glycine substitutions as shown in FIG. 5C (Kabat substitutions 407-410) completely lost the ability to compete with wild type IgG1 (constructs numbered 22 and 24), identifying the Kabat 407-410 region as a requisite for ICAM-1 binding. Assays represent triplicate values and statistics were determined using the Student's T-test.

FIG. 6. A schematic diagram of the IgG1 ICAM-1 binding region is shown in context of the CH3 domain and other functional binding sites for CD16a Fc-γ-receptor, C1q and FcRN binding.

FIG. 7. Bioassay analysis of saporin-linked trastuzumab and pertuzumab (referred to as ZAP) antibodies on killing of HER2⁺ HCT116 wildtype (HCT116-WT) and HCT116 ICAM-1 knockdown (HCT116-ICAM1-KO) cells. As shown, both trastuzumab-ZAP and pertuzumab-ZAP significantly killed HCT116-ICAM1-KO cells in contrast to HCT116-WT cells (P<0.025), demonstrating the negative impact that membrane expressed ICAM-1 can have on internalization and target cell toxicity of antibodies linked to toxins such as ADCs. Assays represent triplicate values and statistics were determined using the Student's T-test.

FIG. 8. ELISA analysis of rituximab containing heavy chain CH3 modifications within the sICAM-1 binding domain. Competitive ELISA assays were run to test for sICAM-1 binding to parental rituximab (RTX), and RTX with modified residues at positions 369-372, 374-377, and 407-410 (referred to as RTX-FARV). As shown, modification of amino acids within these three regions significantly reduces sICAM-1 binding to rituximab (P<0.00065). Assays represent triplicate values and statistics were determined using the Student's T-test.

FIG. 9. Bioassay analysis of parental rituximab (RTX) and RTX-FARV in the presence of sICAM-1. Jurkat-CD16a reporter cells were tested for activation against CD20⁺ Daudi target cells with parental rituximab (RTX) or modified RTX (RTX-FARV) in the presence or absence of 10 μg/mL sICAM-1. As shown, both antibodies activated Jurkat-CD16a signaling while the addition of sICAM-1 significantly suppressed RTX Jurkat-CD16a activation in contrast to RTX-FARV (P=0.0015). Assays represent triplicate values and statistics were determined using the Student's T-test.

### REFERENCE TO AN ELECTRONIC SEQUENCE LISTING

The contents of the electronic sequence listing (sequencelisting.xml; Size: 16,000 bytes; and Date of Creation: Aug. 15, 2022) is herein incorporated by reference in its entirety.

### DETAILED DESCRIPTION OF THE INVENTION

To determine if any of the antibodies employed in antibody-based therapies that employ immune-effector mechanisms for tumor killing are negatively impacted by ICAM-1, we tested for the physical binding of ICAM-1 to those antibodies. We also tested for immunosuppressed humoral immune-effector activity against tumor cells. We found that ICAM-1 can bind IgG1 type antibodies and negatively impact their humoral immune-effector activities. Moreover, ICAM-1 binding can impact the efficacy of antibody drug conjugates (ADCs). Cell surface binding of ICAM-1 to antigen-bound ADCs slows ADC internalization rate, which negatively affects ADC efficacy (see Liao, M Z, et al. Clin Pharmacol & Therapeutics 110:1216-1230, 2021).

We have discovered that the ICAM-1 protein can bind to a specific region in the CH3 domain of human IgG1-type antibodies and suppress their immune-effector activities. Here we show that making modifications to this region (IC1-binding region) can make IgG1 antibodies refractory to ICAM-1 immunosuppression. The use of ICAM-1-binding-modified antibodies in cancer patients whose tumors over-express ICAM-1 enhances or permits their utility for therapy (see Maruo Y et al. Int J Cancer 100:486-490, 2002; Wu M et al. Path Res Pract 10.1016/j.prp.2020.153029; Roland C L et al. Surgery 141:705-707, 2007). As exemplars of this approach, we have generated ICAM-1-binding-modified rituximab (SEQ ID NO: 4 and 5), trastuzumab (SEQ ID NO: 6), pertuzumab (SEQ ID NO: 7), cetuximab (SEQ ID NO: 8) and daratumumab (SEQ ID NO: 9). ICAM-1 binding site modification can be employed to engineer the Fc domain of any IgG1-type antibody to generate ICAM-1 refractory antibodies for improved therapy.

We have shown that modifying the Kabat residues 367-425, preferably Kabat residues 369-410 (IC1-binding domain) within the CH3 domain makes an affected antibody refractory to ICAM-1 immunosuppression. Modifications may, for example, be within residues 369-372, 374-377 and/or 407-410 of an IgG1-type antibody. These ICAM1-refractory (also referred to as IC1-modified) antibodies are capable of overcoming humoral immunosuppression by the ICAM-1 protein. They can be used for treating cancers as well as other ICAM-1 immunosuppressed diseases. While not wanting to be limited to any particular theory or mechanism of action, the applicants believe that the ICAM-1 protein engages with residues within and potentially surrounding the Kabat residues 369-410 of IgG1-type antibodies and disrupts the antibody's mediated humoral immune responses by immune-effector cells and/or the complement system. These disruptions include suppression of the C1q-antibody (classical antibody-complement) complex and/or antibody binding to Fc-γ-activating receptors (CD16a, CD32a, CD64a) on immune-effector cells, such as but not limited to natural killer, dendritic, monocytic and myeloid cells. These disruptions result in the downstream inhibition of CDC, ADCC, as well as ADCP. Changes to other portions of the antibody molecule that are proximal to the IC1-binding domain within the antibody 3-dimensional structure may also affect binding of ICAM-1. Thus, proximity to the IC1-binding domain may be assessed not only with regard to the antibody's primary structure, but also its secondary, tertiary, and quaternary structure.

Moreover, recent work has shown that cell surface proteins that bind to antibodies may negatively impact the efficacy of antibody drug conjugates (ADCs) by reduction of internalization (Nicolaides N C. et al. PloS ONE DOI.org/10.1371/journal.pone.0285161, 2023). The cell surface expression of ICAM-1 may impact ADCs in a similar fashion. Therefore, the use of IC1-modified antibodies may also improve ADC therapeutic efficacy of tumors expressing ICAM-1.

The methods described here may be used to develop additional ICAM-1 refractory antibodies that target tumor associated antigens such as but not limited to CD20, CD38, HER2 or EGFR to overcome humoral immunosuppression or ADC suppression. IC1-modified antibodies that are capable of evading the inhibitory effect(s) of ICAM-1 on humoral immune responses and/or ADC suppression may be used for preclinical and human testing and in clinical practice.

For therapeutic applications, IC1-modified antibodies can be administered with or without other standard-of-care agents. They can be administered prior, during or after administration of the IC1-modified antibody or ADC.

Compositions formed in the course of conducting the methods may be useful diagnostically or therapeutically. The compositions may be combinations of proteins with altered amino acids within the domains affected by other tumor immunosuppressive proteins, such as CA125/MUC1 on rituxumab (SEQ ID NO: 14) (Grasso L, et al. Oncol Letters 23:2, 2022), for example. Other examples include making IC1-modifications to Fc domains containing modifications that can alter ADCC, CDC or FcRN biology, known by those skilled in the art. Any selection of antibodies described here may be used to form diagnostic or therapeutic compositions. The antibodies may also be used analytically as laboratory reagents.

The methods to develop compositions by modifying the IC1-binding domain as described here are useful for any antibody whose dynamic structure is altered by ICAM-1 binding, which leads to suppressed humoral immune responses and/or antibody internalization.

The “dynamic structure” of an antibody or protein is the three-dimensional structure of an antibody at a given time point, wherein such time point coincides with the antibody's engagement with another protein or agent, and the structure of the antibody before this time point has changed into a different structure after this time point in response to the antibody's engagement with the other protein or agent.

Antibody-based approaches continue to be pursued against various cancers as well as inflammatory and infectious diseases, and tumors utilize various pathways to evade host immune defense. Therefore, it is important to identify agents as well as regions within affected antibodies that can be modified to overcome the humoral immunosuppression and/or internalization of affected antibodies. These agents or modified antibody compositions and methods enable the selection of lead antibodies that may overcome the immunosuppression and/or reduced internalization caused by ICAM-1 and be useful for patient screening. For example, patients can be screened to determine if they have elevated ICAM-1. For those patients that do, one can administer an IC1-modified antibody targeting a tumor-specific antigen (i.e., rituximab (CD20 antigen); trastuzumab and pertuzumab (HER2 antigen); cetuximab (EGFR antigen); daratumumab (CD38 antigen), etc.) to overcome ICAM-1's inhibitory effect on humoral responses and/or internalization that may affect ADC formatted antibodies.

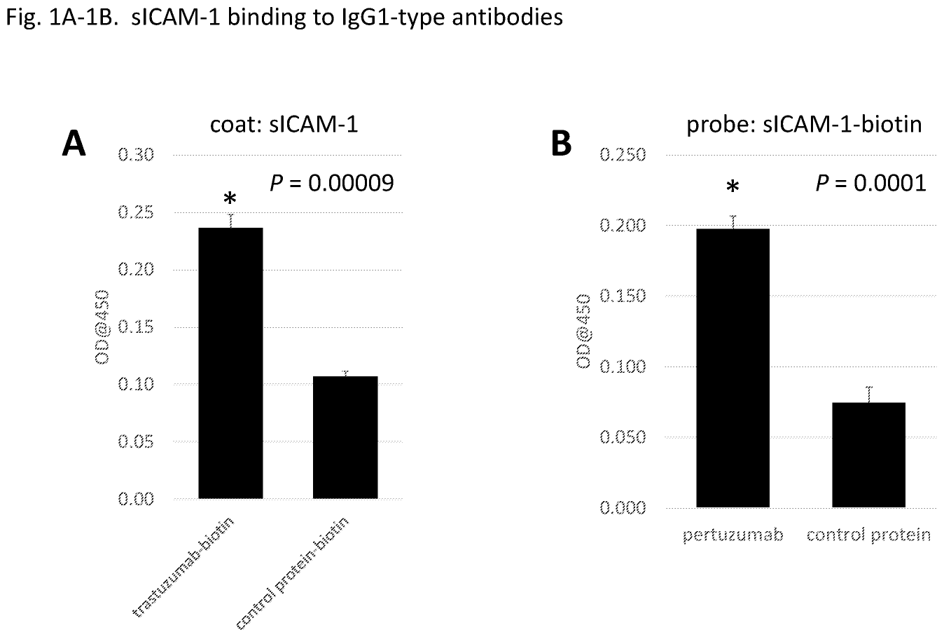

One can screen an IgG1-type antibody's dynamic structure in the presence of ICAM-1 that can affect its dynamic structure and suppress its downstream immune-effector function(s) and/or internalization upon binding to its cell surface target antigen. FIGS. 1A-1B provide an example of screening for ICAM-1 binding and the following examples and figures teach how to modify such ICAM-1 sensitive antibodies to make them refractory to its immunosuppression.

In one embodiment, human cancer cells expressing ICAM-1 can be used to screen for ICAM-1 immunosuppression of a wild type vs. IC1-modified antibody.

In another embodiment, human cancer cells expressing ICAM-1 can be used to screen for antibody internalization rates and/or efficacy of a wild type vs. IC1-modified antibody drug conjugate (ADC).

An IC1-modified antibody may comprise one or more amino acid changes within or in proximity to the IC1-binding domain region (Kabat 369-410 residues) of human IgG1 heavy chain. The proximity may be in the primary, secondary, tertiary, or quaternary structure. The amino acid changes may be within Kabat residues 367-425. An IC1-modified antibody can be tested for the ability to overcome ICAM-1 humoral immunosuppression of ADCC, ADCP or CDC as well as suppressed ADC activity.

In one method for measuring the ability of an IC1-modified antibody to be effective in overcoming ICAM-1 humoral immunosuppression, the antibody is tested for direct ICAM-1 binding and/or the ability of the antibody to bind CD16a or C1q proteins in the presence of ICAM-1 via ELISA or other methods known to those skilled in the art.

In another method used for measuring the ability of an IC1-modified antibody to be effective in overcoming ICAM-1 suppression of an ADC formatted antibody, the antibody is tested for direct ICAM-1 binding and/or the rate of target cell internalization and/or ADC target cell killing in comparison to an IC1-modified format using methods known to those skilled in the art.

Functional methods can be used to measure the effect of IC1-modified antibody on overcoming humoral immunosuppression by ICAM-1 using ADCC, ADCP or CDC. ICAM-1 can be produced from a target cell or added as an exogenous “agent” in soluble form. The term “effect” generally refers to a 10% or greater change in ADCC, ADCP or CDC target cell killing when an agent is incubated with parental versus the IC1-modified antibody. It may, depending on the antibody, refer to a change of at least 5%, 15%, 20%, 25%, 30%, 35%, 40%, 45%, 50%, 55%, 60%, 70%, or 75%.

Various terms and terminology relating to aspects of the enclosed description are used throughout the specification and claims of this document. Such terms are to be given their ordinary meaning in the art unless otherwise specifically indicated. Other specifically defined terms are to be construed in a manner consistent with the definitions provided.

As used in this specification and the appended claims, the singular forms of “a,” “an,” and “the” also include plural references unless the content clearly specifically dictates otherwise. As example, reference to “a cell” may include a combination of two or more cells, and the like. Reference to “a probe” may include the parental or IC1-antibody, or an independent antibody to an antigen to monitor humoral immune response via any analytical method known by those skilled in the art.

The term “about” as used when referring to quantified values such as an amount, a period of time, and/or the like, is meant to encompass variations of up to +9% from the specified value, as such variations are appropriate to carry out the disclosed methods. Unless otherwise indicated, all values expressing quantities of reagents, such as molecular weight, molarity, reaction conditions, percentage and so forth used in the specification and claims are to be understood as being quantified in all instances by the term “about.” Accordingly, unless indicated to the contrary, the numerical values as set forth in the following specifications and listed claims are approximations that may vary depending upon the desired properties of the composition agent and/or methods sought to be obtained by the present invention. At the very least, and not as an attempt to limit the scope of the application, each numerical value should at least be valued by the reported significant digits and through ordinary rounding methods known by those skilled in the art.

The term “antibody” as used here is meant in a broad sense and includes immunoglobulin (also referenced as “Ig”) or antibody molecules including polyclonal antibodies (also referenced as pAbs), monoclonal antibodies (also referenced as mAbs) including murine, human, humanized and chimerized mAbs, bispecific antibodies (also referenced as BSPs), and antibody fragments, and antibody drug conjugates (also referred to as ADCs). In general, antibodies are proteins or polypeptide chains that bind to a specific antigen. An antigen is a structure that is specifically recognized by a given antibody. Canonical antibodies comprise a hetero-tetramer of glycosylated proteins, composed of two light chains and two heavy chains lined through a complex of disulfide and hydrogen bonds. The term “its disulfide bridge” refers to the disulfide bridge contained within the heavy chain hinge region, which is commonly known by those skilled in the art. Each heavy chain has a variable domain (variable region) (VH) followed by a number of constant domains referred to as CH1, CH2 and CH3 that make up the Fc domain. Each light chain has a variable domain (VL) and a constant domain; the constant domain of the light chain is aligned with the first constant domain of the heavy chain and the light chain VL is aligned with the variable domain of the heavy chain. Antibody light chains of any species are assigned to one of two distinct types based on their amino acid sequences within their constant domains, namely kappa (κ) and lambda (2).

Immunoglobulins are categorized as classes or isotypes, depending upon the type of Fc domain namely IgA, IgD, IgE, IgG and IgM, which depend on the sequences contained within their heavy chain constant domain. The IgA and IgG isotypes are further comprised of subclasses as the isotypes IgA₁, IgA₂, IgG₁, IgG₂, IgG₃ and IgG₄.

An immunoglobulin VL or VH region consists of a “framework” (FW) region interrupted by three “antigen-binding sites” also referred to as Complementarity Determining Regions (CDRs) based on sequence variability as reported (Wu T T, Kabat E A. J Exp Med 132:211-250, 1970). In general, an antigen-binding site is composed of six CDRs with three located within the heavy chain (CDRH1, CDRH2, CDRH3), and three within the light chain (CDRL1, CDRL2, CDRL3) variable domains (Kabat E A, et al. 5th Ed. PHS, National Institutes of Health, Bethesda, Md., 1991).

“Kabat” numbering refers to a scheme for the numbering of amino acid residues in antibodies based upon variable regions.

“Specific binding” or “specifically binds” refers to the binding of an antibody or antigen-binding fragment to an antigen (including sequences contained within an antibody itself) with greater affinity than for other antigens. Typically, a specific antibody or antigen-binding fragment binds target antigen with an equilibrium dissociation constant K_{D} of about 5×10⁻⁸ M or less.

An “antigen” is an entity to which an antibody or antibody fragment specifically binds. This includes binding to an antibody or protein of interest.

The term “antibody dynamic structure” refers to any change in structure that can affect antibody humoral function (i.e., Fc receptor or C1q binding, etc.).

The term “monoclonal antibody (mAb)” refers to an antibody that is derived from a single cell clone, including any eukaryotic or prokaryotic cell clone, or a phage clone, and not the method by which it is produced. Thus, the term “monoclonal antibody” is not limited to antibodies produced through hybridoma technology but may also include recombinant methods.

“Full length antibody” refers to an IgG1 type antibody containing full variable and heavy chain domains as typically found in rodent or human serum or produced by recombinant DNA methods.

“Fab domain” refers to any antibody sequence N-terminal to the antibody hinge disulfide region which is known by those skilled in the art.

“Fc domain” refers to any antibody sequence C-terminal to the antibody Fab domain and includes the antibody hinge disulfide region which is known by those skilled in the art.

The “affected domain” or “ICAM-1 binding domain” or “IC1-region” refers to the amino acid sequences located in and around the human IgG1 Kabat 369-410 region, for example, within Kabat 407-410 (SEQ ID NO: 3) region. The affected domain may be within the CH3 domain, i.e., Kabat residues 367-425.

The term “parental antibody” refers to a human IgG1-type antibody composed of wild type IgG1 heavy chain sequences.

The term “IC1-modified antibody” or “IC1-antibody” refers to an antibody composed of a heavy chain containing one or more amino acid changes within or in proximity to the ICAM-1 binding domain. Substitutions within the Kabat region 367-425 (CH3 domain) or Kabat region 369-410, including region 407-410 may utilize any amino acid, including but not limited to alanine (ala—A), arginine (arg—R), asparagine (asn—N), aspartic acid (asp—D), cysteine (cys—C), glutamine (gln—Q), glutamic acid (glu—E), glycine (gly—G), histidine (his—H), isoleucine (ile—I), leucine (leu—L), lysine (lys—K), methionine (met—M), phenylalanine (phe—F), proline (pro—P), serine (ser—S), threonine (thr—T), tryptophan (trp—W), tyrosine (tyr—Y), and valine (val—V). One, two, three, or four of the residues in the Kabat 367-425 region can be substituted.

The term “ICAM-1-refractory” refers to an IC1-modified antibody that has better ADCC, ADCP, CDC and/or internalization for ADC formats than the parental antibody.

The term “agent” refers to any protein or chemical able to block or reduce antibody immune-effector function.

The term “affected antibody” refers to an antibody whose humoral immune function or internalization/antibody drug conjugate target cell killing is reduced by ICAM-1.

The term “rituximab” refers to the FDA approved antibody [FDA Reference ID: 4274293].

The term “cetuximab” refers to the FDA approved antibody [FDA Reference ID: 4422941].

The term “trastuzumab” refers to the FDA approved antibody [FDA Reference ID: 4090445].

The term “pertuzumab” refers to the FDA approved antibody [FDA Reference ID: 3384285].

The term “daratumumab” refers to the FDA approved antibody [FDA Reference ID: 4924146].

The term “CD20” refers to the human cell surface protein expressed by B-cells and is the target antigen to which rituximab specifically binds.

The term “HER2” refers to the human cell surface protein expressed by epithelial cells and is the target antigen to which trastuzumab and pertuzumab specifically bind.

The term “EGFR” refers to the human cell surface protein expressed by epithelial cells and is the target antigen to which cetuximab specifically binds.

The term “CD38” refers to the human cell surface protein expressed by lymphoid cells and is the target antigen to which daratumumab specifically binds.

The term “CA125” refers to the gene product produced by MUC16 gene (HGNC: 15582; OMIM: 606154), which is found in soluble and membrane-bound forms. It binds to antibodies and affects bound antibody humoral immune function (Kline J B et al. Oncotarget 8:52045-52060, 2017).

The term “ICAM-1” refers to the gene product produced by the ICAM-1/CD54 gene (HGNC: 5344; NCBI Reference Sequence: NG_012083.1), which is found in soluble and membrane-bound forms. It binds to CD11a/CD18 and CD11b/CD18.

The terms “cancer,” “malignant,” “dysregulated,” and “tumor” are well known in the art and refer to the presence of cells with unregulated cell growth and morphological features different than a normal cell type of similar origin. Malignant refers to those cancer cells capable of causing morbidity and/or mortality. As used here, “cancer” and “tumor” include premalignant and malignant types.

As used here, the term “soluble” refers to a protein or non-protein agent that is not attached to the cellular membrane of a cell. For example, an agent that is soluble may be shed, secreted or exported from normal or cancerous cells into biological fluids including serum, whole blood, plasma, urine or microfluids of a cell, including tumor cells.

The “level” of a specified protein agent including IC1-modified antibodies and ICAM-1, as used, refers to the amount of the agent as determined using any method known in the art for the measurement of protein levels in vitro or in vivo. Such methods include gel electrophoresis, capillary electrophoresis, high performance liquid chromatography (HPLC), thin layer chromatography (TLC), hyperdiffusion chromatography, fluid or gel precipitation reactions, absorption spectroscopy, colorimetric assays, spectrophotometric assays, flow cytometry, immunodiffusion (single or double), solution phase assay, immunoelectrophoresis, Western blotting, radioimmunoassay (RIA), enzyme-linked immunosorbent assays (ELISAs), immunofluorescent assays, fluorescence resonance energy transfer (FRET), Förster resonance energy transfer, electrochemiluminescence immunoassay, and the like. In one embodiment, the level of ICAM-1 is determined using probe-based techniques.

The term “humoral immuno-suppression,” humoral immunosuppression,” or humoral immune suppression” refers to any antibody, antibody fragment, bispecific antibody (BSP) or antibody drug conjugate (ADC) that is directly bound by ICAM-1 and whose dynamic structure is altered. ICAM-1 can be in membrane-bound form or soluble form.

The term “bispecific antibody (BSP)” refers to any antibody that can bind two or more different antigens. A BSP can comprise at least but not limited to two full-length antibodies, a full-length antibody and a single chain antibody, or two single chain antibodies, wherein each one binds to different antigens or different epitopes on the same antigen.

The term “antibody dependent cellular cytotoxicity (ADCC)” refers to an in vitro or in vivo process in which an antibody can bind to an antigen on a surface of a cell then engage with immune-effector cells via sequences within the antibody's Fc domain that in turn results their release of toxins that can kill bound cell.

The term “complement dependent cytotoxicity (CDC)” refers to an in vitro or in vivo process in which an antibody can bind to an antigen on a surface of a eukaryotic or prokaryotic cell then engage with the C1q protein via sequences within the antibody's Fc domain that in turn results in initiation of classical complement cascade that can kill bound cell.

The term “opsonization” refers to a process called antibody dependent cellular phagocytosis (ADCP) where an antibody can bind to an antigen on a surface of a cell then engage with immune cells via sequences within its Fc domain that in turn results in immune cells engulfing, consuming and ultimately killing antibody bound cell.

The term “native” or “naïve” or “canonical” antibody or “antibody format” refers to a parental or IC1-modified antibody that is full length without any chemical modifications.

The term “antibody drug conjugate (ADC)” refers to an antibody that is covalently or non-covalently linked to a chemical or a biological agent. These include radionuclides, nucleic acids, immunotoxins, small molecular weight compounds. Linked agents may be cytotoxic, cytostatic, or diagnostic (emit detectable signals via fluorescence, luminescence, radiographic or enzymatic signals).

The term “pharmacokinetic (PK)” refers to the time that an antibody maintains its steady-state concentration when administered to a subject.

The term “pharmacodynamic (PD)” refers to the study of the biochemical and physiological effects of an antibody-based drug and its mechanisms of action(s), including the correlation of their actions and effects with their biochemical structure when administered to a subject.

The term “pharmacologic (PL)” refers to the known effect an antibody has on managing or killing a disease cell in vitro or in vivo.

The term “sample” refers to a collection of similar fluids, cells or tissues isolated from a subject, as well as fluids, cells or tissues present within a subject. Fluids may include biological fluids that include liquid solutions contacted with a subject or biological source, including cell and organoid culture medium, urine, salivary, lavage fluids and the like.

The term “control sample” or “control protein” as used, refers to any clinically or non-clinically relevant control sample, including, for example, a sample from a healthy subject not afflicted with a particular cancer type or a cell that is different from its parental cell.

The term “control level” refers to an accepted or pre-determined level (amount) of a protein or non-protein agent that is used to compare with the level of the same agent in a sample derived from a subject or used in in vitro assays.

As used, “a difference” between signal of an antibody in control settings vs being bound by ICAM-1 is generally any difference that can be determined using statistical methods commonly used by those skilled in the art and at a minimum a difference of 10% or greater as compared to control. It may, depending on the antibody and the probes used, also refer to a change of at least 5%, 15%, 20%, 25%, 30%, 35%, 40%, 45%, 50%, 55%, 60%, 70%, or 75%.

The term “inhibit,” “inhibition of,” “suppress,” or “reduce” means to reduce by a statistically measurable amount, or to prevent entirely.

The term “functional,” in the context of an antibody or antibody containing moiety (i.e. ADC, BSP, etc.) to be used in accordance with the methods described, indicates that the IC1-modified antibody has reduced ICAM-1 binding to an IgG1-type antibody, respectively, and/or is able to kill target cells in vitro or in vivo better than parental antibody alone.

The term “target cell” refers to a eukaryotic or prokaryotic cell or population of cells that expresses an antigen for a specific antibody or antibody containing moiety.

The term “pharmaceutically acceptable” refers to a substance that is acceptable to administer to a patient from a pharmacological as well as toxicological aspect and is manufactured using approaches known by those skilled in the art. These include agents approved by a regulatory agency of the Federal or a state government or listed in the U.S. Pharmacopeia or other generally recognized pharmacopeia for use in animals and humans. The term “pharmaceutically compatible ingredient” refers a pharmaceutically acceptable diluent, adjuvant, excipient or matrix vehicle with which an anti-cancer agent is administered. “Pharmaceutically acceptable carrier” refers to a matrix that does not interfere with the effectiveness of the biological activity of the active ingredient(s) and is nontoxic to the host.

The terms “effective amount” and “therapeutically effective” are used interchangeably and, in the context of administering a pharmaceutical agent at an amount that is sufficient to produce an enhanced clinical outcome in a patient. An effective amount of an agent is administered according to the methods described here in an “effective regimen.” The term “effective regimen” refers to a combination of amount of the agent and dosage frequency adequate to accomplish an enhanced clinical outcome for a patient with a particular cancer. Enhanced efficacy is an improved clinical outcome when a patient is administered an agent that is capable of overcoming morbidity better than a parental compound or an agent that can enhance the clinical outcome of an effective regimen. As in context here, effective amount refers to the amount of IC1-modified antibody required to demonstrate efficacy or a difference when compared to parental antibody.

The terms “patient” and “subject” are used interchangeably to refer to humans and other non-human animals, including veterinary subjects, that receive a therapeutic agent treatment. The term “non-human animal” includes all vertebrates. In one embodiment, the subject is a human.

“Therapeutic agents” are typically substantially free from undesired contaminants. This means that an agent is typically at least about 50% w/w (weight/weight) pure as well as substantially free from interfering proteins and contaminants.

The term “immune-effector cell” refers to any cell including but not limited to natural killer (NK), myeloid, monocytic, and/or dendritic cells that may confer antibody dependent cellular cytotoxicity (ADCC) or phagocytosis (ADCP, opsonization) upon binding to antibody-bound target cell. Cells may be purified or present in mixture in the form of peripheral blood mononuclear cells (PBMCs).

Inflammatory diseases include auto-immune diseases, as well as rheumatoid arthritis, granulomatosis with polyangiitis, idiopathic thrombocytopenia purpura, pemphigus vulgaris, myasthenia gravis, atherosclerosis, and Epstein-Barr virus-positive mucocutaneous ulcers.

Infectious diseases include viral, bacterial, protozoan, and parasitic diseases. Viral diseases include but are not limited to influenza, AIDS, meningitis, pneumonia, herpes, human papillomavirus, and respiratory syncytial virus, parainfluenza, ebola, measles, chickenpox, and shingles. Bacterial diseases include but are not limited to tuberculosis, pertussis, legionnaires' disease, pneumonia, and urinary tract infections. Protozoan diseases include malaria, giardia, Chagas disease, amoebiasis, trichomoniasis, trypanosomiasis, and toxoplasmosis. Parasitic diseases include without limitation filariasis, pediculosis, fasciola, schistosomiasis, and cryptosporidoiosis.

The term “dysregulated cell” refers to any cell that is deemed abnormal relative to parental cells. These include transformed cells, malignant cells, virally infected cells, autonomously growing cells via autoregulation.

The term “test antibody” refers to antibody be analyzed for immune effector activity.

The term “humoral response” refers to ADCC, ADCP and/or CDC activity by test antibody.

The term “screening” may refer to testing of proteins that can bind to ICAM-1 in the presence of an affected antibody or antibody-containing moiety (i.e., BSP, ADC, single chain antibody, antibody fragment, etc.) and looking for enhanced biological response monitoring total cell or ADCC, ADCP or CDC mediated killing. The term may be used in other contexts in which a large number of test elements are being assayed to determine which among the test elements has a certain property. Similarly, it can be used to refer to the assaying of patient samples for those having a particular property, such as elevated ICAM-1 RNA or protein.

The term “significant(ly)” refers to statistical results where the P value as determined by any number of programs including the Student's T-Test is less than 0.05.

### Composition of Modified Therapeutic Antibodies and Methods For Developing and Overcoming ICAM-1/CD54-Mediated Humoral Immune Suppression and Reduced Antibody Drug Conjugate Efficacy.

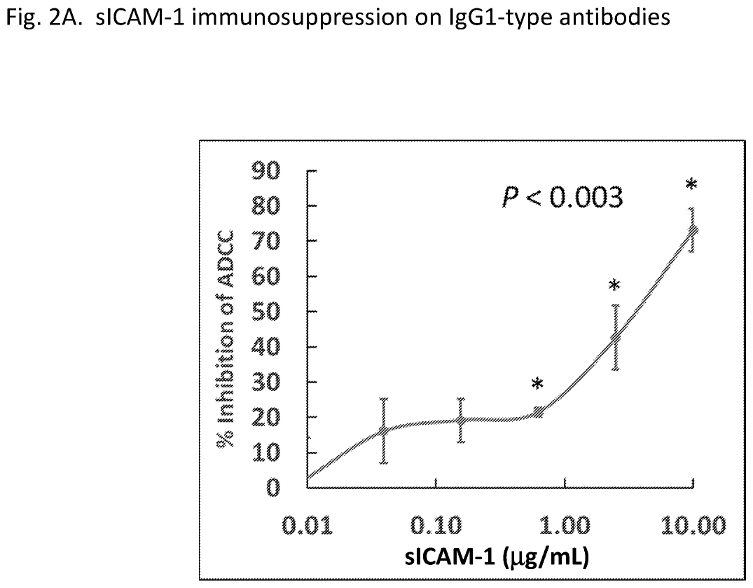

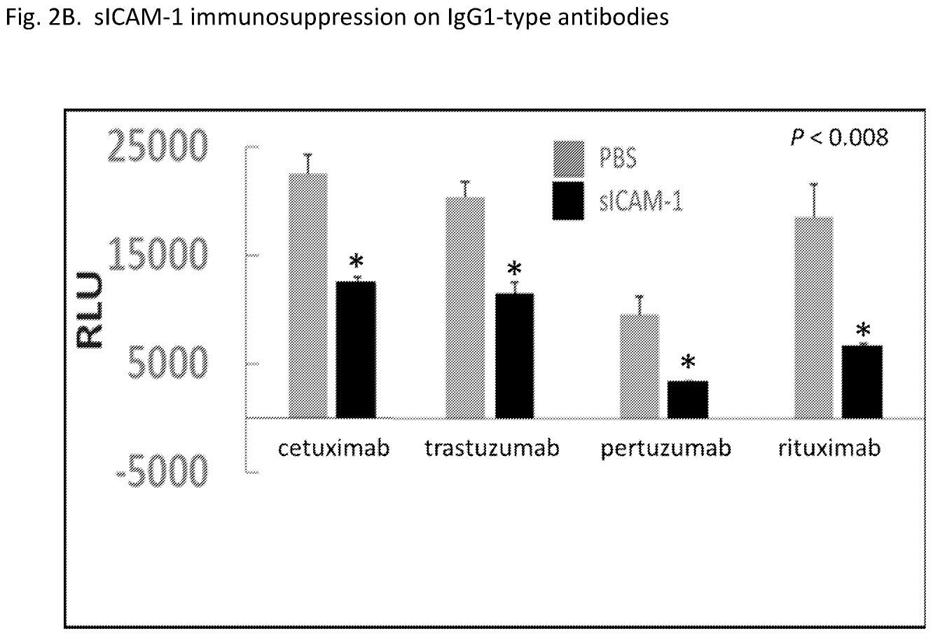

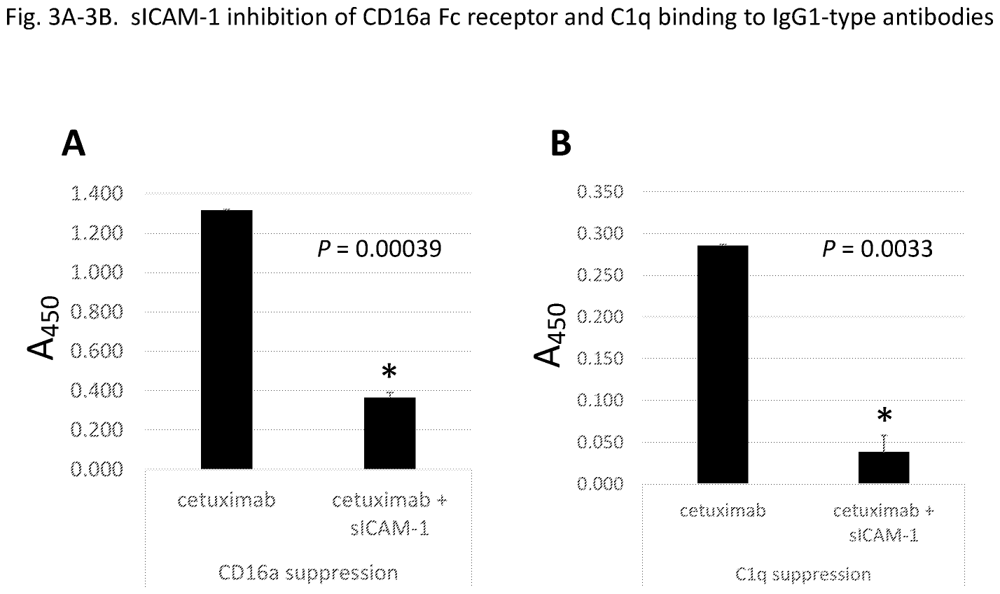

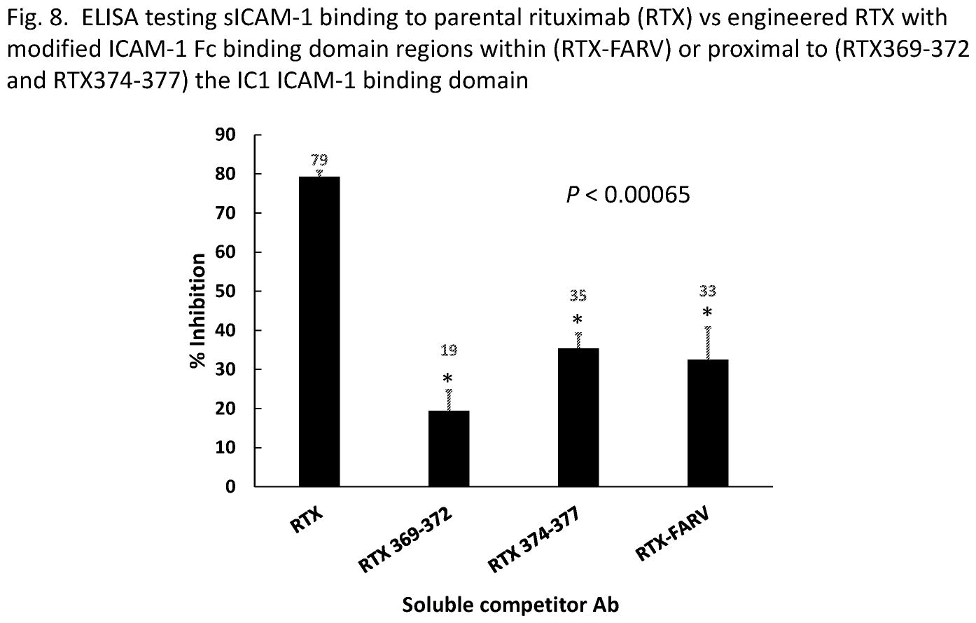

ICAM-1/CD54 binding to human IgG1-type antibodies results in reduced immune-effector activities. Engineered IgG1-type antibodies can overcome ICAM-1/CD54 suppression of humoral immune response and suppression of antibody drug conjugate (ADC) efficacy by affected parental IgG1 antibodies. In some embodiments, the methods for identifying ICAM-1/CD54 refractory antibodies involve generating IgG1-type antibodies containing one or more amino acid changes within or around the affected ICAM-1/CD54 binding domain (IC1-region) and testing for the ability of the IC1-modified IgG1 antibody to have significantly improved biological activity when used to mediate ADCC, ADCP or CDC killing or improved ADC target cell killing against antigen-specific expressing target cells. In some embodiments, the IgG1 antibody consists of one amino acid change. In other embodiments, the IgG1 antibody consists of two or more amino acid changes. Optimal amino acid changes can be determined using functional ADCC, ADCP, CDC, ADC killing assays in the presence of an antibody with mutated amino acids within or in proximity to the IC1-region. These assays can employ primary cells or reporter cells, such as the Jurkat-CD16a ADCC reporter cell line. Examples are provided in Example 2 and shown in FIG. 2 and FIG. 8. Identification of ICAM-1-refractory IgG1 antibodies that are able to significantly overcome humoral immunosuppression by ICAM-1/CD54 can also be identified by employing molecular assays to monitor CD16a Fc receptor or C1q protein binding to antibody via assays commonly used by those skilled in the art as shown in FIG. 3.

An IC1-modified antibody may have a binding affinity similar or greater than the parental IgG1 antibody.

In some methods for identifying ICAM-1/CD54-refractory IgG1 antibodies, antibody is added to a culture of target cells in which target cells naturally or recombinantly express an IgG1 specific antigen and ICAM-1. Cultures for comparing humoral responses in the presence of IC1-modified vs parental antibody can be monitored using standard CD16a-activation, ADCC, ADCP or CDC killing assays. A change in at least 10% is typically considered as being a meaningful effect on ADCC and/or CDC functions. Depending on the antibody and the assay employed, a meaningful effect also may be defined as a change of at least 5%, 15%, 20%, 25%, 30%, 35%, 40%, 45%, 50%, 55%, 60%, 70%, or 75%. The target cell line may be engineered to over-express or have reduced ICAM-1 expression via transduction with a human ICAM-1 expression construct or an ICAM-1 shRNA knock-down line via shRNA specific constructs (SEQ ID NO: 10-13), respectively.

A cancer subject may be treated with a IC1-modified IgG1 antibody. For example, a patient may have a CD20-expressing cancer such as, but not limited to, Hodgkin's Lymphoma, Non-Hodgkin's Lymphoma, Follicular Lymphoma, Large Cell Lymphoma, or Chronic Lymphocytic Leukemia. Several reports have found ICAM-1 expression elevated in patients with such cancers to have poor prognosis. Rituximab is a standard-of-care for a subset of these cancer types. In such a case, an IC1-modified rituximab antibody may be a desirable entity. The IC1-modified antibody may be formatted as an antibody drug conjugate or in native state.

Over-expression of ICAM-1 in breast and gastric cancers have been reported to lead to worse prognosis. Both the anti-HER2 trastuzumab and pertuzumab IgG1 antibodies have been approved to treat these cancer indications. In some embodiments, an IC1-modified trastuzumab or pertuzumab may be a desirable entity to treat breast cancer or gastric cancer patients over-expressing ICAM-1. The IC1-modified antibody may be formatted as an antibody drug conjugate or in its native state.

An IC1-modified cetuximab that targets the EGFR protein may be used therapeutically. Cetuximab is approved for the treatment of colorectal cancer and head and neck cancer. Patients afflicted with these cancer indications and exhibit elevated ICAM-1 can benefit from an IC1-modified cetuximab, thereby making an IC1-modified cetuximab a desirable entity. The IC1-modified antibody may be formatted as an antibody drug conjugate or in its native state.

An IC1-modified daratumumab that targets the CD38 protein may be used therapeutically. Daratumumab is approved for the treatment of multiple myeloma. Patients afflicted with these cancer indications and exhibit elevated ICAM-1 can benefit from a IC1-modified daratumumab, thereby making an IC1-modified daratumumab a desirable entity. The IC1-modified antibody may be formatted as an antibody drug conjugate or in its native state.

A patient with a CD20, CD38, HER2 or EGFR-positive cancer that over-expresses ICAM-1/CD54 may be treated with an IC1-modified rituximab, daratumumab, trastuzumab/pertuzumab, or cetuximab antibody, respectively, alone in a native or ADC format or in combination with standard-of-care therapy. In some embodiments of the methods of treating a subject with an ICAM-1 over-expressing cancer described here, an IC1-modified antibody is administered to the subject, where the subject has a baseline ICAM-1/CD54 level that is above the normal range. In some embodiments of the methods of treating a subject with ICAM-1 over-expressing cancer described here, the method involves administering the IC1-modified antibody alone. In yet another embodiment, the IC1-modified antibody is administered in combination with chemotherapy. The chemotherapy may be any chemotherapeutic agent considered standard-of-care for a particular cancer indication at the time when the subject is treated. In the methods of treatment described here, ICAM-1/CD54 expression level may be determined by any means known in the art and defined as within or above the normal range by those skilled in the art.

An IC1-modified antibody may have one amino acid change in the affected domain (Kabat region 369-410). The IC1-modified antibody may have two or more amino acid changes in the affected domain. The IC1-modified antibody may be administered to patients with tumors that over-express ICAM-1/CD54 and that express an antigen that is targeted by the antibody. Exemplary cancers known to over-express ICAM-1/CD54 are Hodgkin's and Non-Hodgkin's Lymphoma, Follicular Lymphoma, Large Cell Lymphoma, and Chronic Lymphocytic Leukemia. Other cancers may also be amenable to such treatment, including, without limitation, multiple myeloma, melanoma, breast, lung, colorectal, gastro-intestinal, and head and neck cancers.

The present methods can be combined with other means of treatment such as surgery (e.g., debulking surgery), radiation, targeted therapy, chemotherapy, immunotherapy, use of growth factor inhibitors, or anti-angiogenesis factors. An IC1-modified antibody can be administered concurrently to a patient undergoing surgery, chemotherapy or radiation therapy treatments. Alternatively, a patient can undergo surgery, chemotherapy or radiation therapy prior to or subsequent to administration of the IC1-modified antibody by at least an hour and up to several months, for example at least an hour, five hours, 12 hours, a day, a week, a month, or three months, prior or subsequent to administration of standard of care therapy. In some cases, one can administer a therapeutically effective amount of chemotherapy plus an IC1-modified antibody, such as rituximab, daratumumab, trastuzumab, pertuzumab, cetuximab, etc.

A subject may have received first-line surgical resection of the tumor or first-line chemotherapy for treatment of the cancer prior to administering an IC1-modified antibody specific to an antigen expressed by said cancer.

A therapeutic antibody may be used that comprises the CDR sequences that can direct binding of an antibody to the CD20 antigen and contain an IC1-modified heavy chain (e.g., SEQ ID NOs: 4 or 5); an antibody to the HER2 antigen and contain an IC1-modified heavy chain (e.g., SEQ ID NOs: 6 or 7); an antibody to the EGFR antigen and have a IC1-modified heavy chain (e.g., SEQ ID NO: 8); an antibody to the CD38 antigen and have an IC1-modified heavy chain (e.g., SEQ ID NO: 9) each of which contain one or more modifications within or in proximity to the ICAM-1/CD54 binding domain (Kabat region 369-410). The antibody may be in an ADC or native format. The appropriate antibody may be administered to a subject with an antigen-positive (relative to the therapeutic antibody) disease indication and a level of ICAM-1/CD54 that is above the normal range. Treatment can optionally include surgery as well as treatment with standard-of-care.

Various delivery systems can be used to administer the IC1-modified antibody including intradermal, intramuscular, intraperitoneal, intravenous, and subcutaneous routes as deemed necessary. Antibodies can be administered, for example by infusion or bolus injection, by absorption through epithelial or mucocutaneous linings via systemic or local approaches. Typically, the antibody is infused intravenously.

The IC1-modified antibody can be administered by injection via syringe, catheter, or any implantable matrix or device.

The IC1-modified antibody in combination with other drugs can be administered as pharmaceutical compositions comprising a therapeutically or prophylactically effective amount of the therapeutic agent(s) and one or more pharmaceutically acceptable or compatible ingredients.

The amount of the therapeutic agent that is effective in the treatment or prophylaxis of a cancer or non-oncologic disease can be determined by standard clinical techniques. In addition, in vitro assays may optionally be employed to help identify optimal dosage ranges required for the IC1-modified antibody. Effective doses may be extrapolated from dose-response curves of IC1-modified antibody derived from in vitro or animal model test systems.

For example, toxicity and therapeutic efficacy of the IC1-modified antibody can be determined in cell cultures or experimental animals by standard pharmaceutical procedures for determining the LD₅₀ (the dose lethal to 50% of the population) and the ED₅₀ (the dose therapeutically effective in 50% of the population) values. The dose ratio between toxic and therapeutic effects is the therapeutic index and it can be expressed as the ratio LD₅₀/ED₅₀. Agents that exhibit large therapeutic indices are suitable. When an agent exhibits toxic side effects, a delivery system that targets the agent to the site of affected tissue can be used to minimize potential damage to non-antigen-expressing cells and, thereby, reduce side effects.

Nucleic acid vectors may be plasmids, viruses, or subviral, for example. Preferably the nucleic acid will be maintained in a desired host cell by having an effective origin of replication, but in some situations, transient expression may be desired. Typically, it will be desirable to have the IC1-modified antibodies expressed, requiring expression control sequences on the vector and appropriate accessory proteins in the host cell. Often, to produce large quantities of an antibody or antibody fragment, stable cell lines will be formed that express all or a fragment of the antibody. Antibody fragments may be entire light or heavy chains, for example. The nucleic acids and vectors may be used to create cell lines that express the desired antibodies. The cell lines may be used in a manufacturing process. Alternatively, cell lines that express antibodies may be delivered in such a manner that they secrete the antibodies directly to the patient. For example, the cell lines may be in a subcutaneous device that manufactures protein in situ. Alternatively, the nucleic acids or vectors may be delivered to the subject in a form of genetic therapy.

The dosing and dosage schedule may vary depending on the active drug concentration, which may depend on the needs, size, and status of the subject.

According to one aspect of the invention, a method of treating a cancer patient or a patient with an inflammatory disease is provided. An ICAM-1-modified (referred to herein as IC1) human IgG1-type antibody is administered to the cancer patient or the patient with an inflammatory or infectious disease. The administered protein is refractory to ICAM-1 binding and enhances the effect of a therapeutic antibody with or without an additional modified Fc domain. The IC1-modified antibody may contain of 1, 2, 3, or 4 amino acid modifications or a combination of amino acids that change the sequence of wild-type human IgG1 Fc domain within the IC1-binding domain (Kabat residues 369-410) or have a deletion of a portion of the IC1 binding domain. The antibody may have one or more amino acid changes within or outside of the IC1-binding domain (Kabat region 369-410). Any antibody containing a modification of the 367-425 region, including or excluding modifications within Kabat region 407-410 is referred to here as an IC1-engineered or IC1-modified antibody.

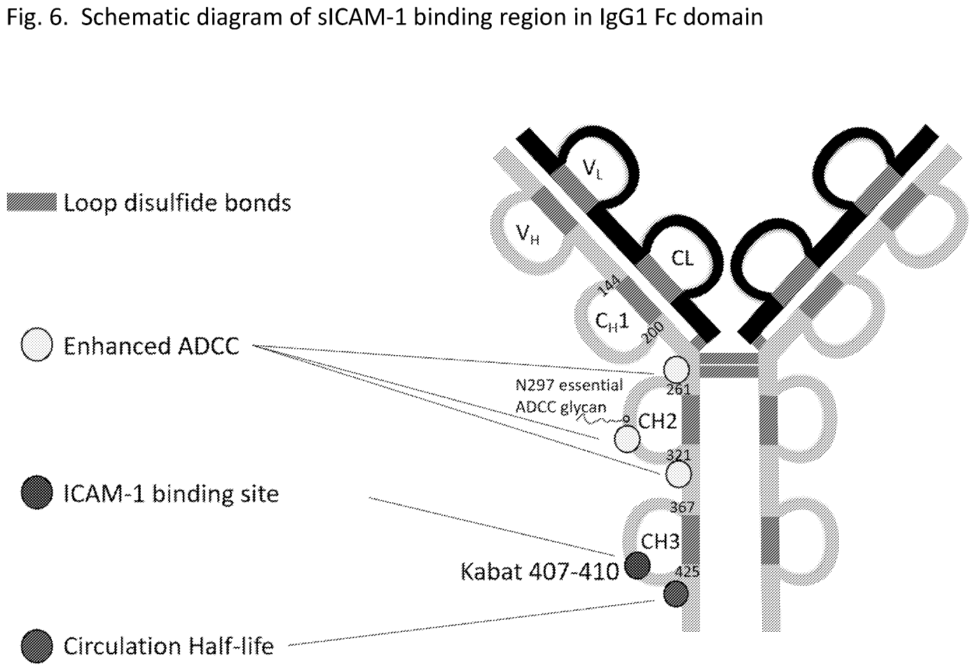

Another aspect of the invention involves modifying one or more amino acids in the human IgG1 CH3 domain (FIG. 6); the modified amino acids reduce ICAM-1 binding yet retain the ability to bind CD16a Fc receptor and C1q protein.

An IC1-engineered IgG1-type antibody targeting a tumor protein can be generated. The tumor protein may be, but is not limited to BCMA, CD19, CD20, CD22, CD30, CTLA-4, CD38, epidermal growth factor, fibroblast growth factor 1, 2, 3 or 4, folate receptor alpha, HER2, mesothelin, PD-1, or PD-L1. All of these tumor proteins are known in the art.

A patient with cancer or an inflammatory disease who expresses an elevated level of SICAM-1 or membrane-bound ICAM-1 compared to a population of healthy humans may be treated. The treatment comprises an IC1-modified antibody targeting an antigen specific to the disease cell. Typically, the agent can be administered daily, once a week, once every 3 weeks, or monthly.

A patient with Non-Hodgkin's Lymphoma (NHL) can be treated with an IC1-modified rituximab containing the wild type light chain and an IC1-modified heavy chain (SEQ ID NO: 4) or a CA125-refractory rituximab (Grasso L et al. Oncol Letters 23:2, 2022) (SEQ ID NO: 5) heavy chain. The IC1-modified rituximab antibody can be administered alone or in combination with other chemotherapeutic agents to the patient. The antibody binds to CD20-positive target cells and elicits a humoral immune response, such as ADCC, ADCP or CDC, to kill the bound cell. The antibody can be in an ADC or a native format.

A patient with a HER2-positive breast, gastric or head and neck cancer can be treated with an IC1-modified trastuzumab containing a wild type light chain and the IC1-modified heavy chain (SEQ ID NO: 6). The IC1-modified trastuzumab antibody can be administered to the patient alone or in combination with other chemotherapeutic agents. The antibody binds to HER2-positive target cells and elicits a humoral immune response, such as ADCC, ADCP or CDC, to kill the bound cell. The antibody can be in an ADC or a native format.

A patient with a HER2-positive breast, gastric or head and neck cancer can be treated with an IC1-modified pertuzumab containing a wild type light chain and the IC1-modified heavy chain (SEQ ID NO: 7). The IC1-modified pertuzumab antibody can be administered to the patient alone or in combination with other chemotherapeutic agents. The antibody binds to HER2-positive target cells and elicits a humoral immune response, such as ADCC, ADCP or CDC, to kill the bound cell. The antibody can be in an ADC or a native format.

A patient with colorectal or head and neck cancer can be treated with an IC1-modified cetuximab containing wild type light chain and the IC1-modified heavy chain (SEQ ID NO: 8). The IC1-modified cetuximab antibody can be administered to the patient alone or in combination with other chemotherapeutic agents. The antibody binds to EGFR-positive target cells and elicits a humoral immune response, such as ADCC, ADCP or CDC, to kill the bound cell. The antibody can be in an ADC or a native format.

A patient with multiple myeloma can be treated with an IC1-modified daratumumab containing a wild type light chain and the IC1-modified heavy chain (SEQ ID NO: 9). The IC1-modified daratumumab antibody is administered to the patient alone or in combination with other chemotherapeutic agents. The antibody binds to CD38-positive target cells and elicits a humoral immune response, such as ADCC, ADCP or CDC, to kill the bound cell. The antibody can be in an ADC or a native format.

In the following examples we show that ICAM-1 (SEQ ID NOS: 1 and 2) can directly bind to IgG1-type immunoglobulins and suppress their immune-effector function. Moreover, we identify a specific region for ICAM-1 binding (referred to the “IC1”-binding region) on the human IgG1 Fc domain, that when modified makes susceptible immunoglobulins refractory to the soluble ICAM-1 (SICAM-1) (SEQ ID NO: 2) immunosuppression. Moreover, residues proximal to the IC1-binding region can also be modified to create an ICAM-1 refractory antibody.

Binding of IgG1 to membrane-bound ICAM-1 (mICAM-1) (SEQ ID NO: 1) may participate in inflammatory disease. The IC1 modifications can be made to wild type IgG1 Fc domains or IgG1 Fc domains with optional additional modifications to enhance or decrease ADCC, ADCP and/or CDC activities (Natsume A, et al. Drug Design, Devel Therapy 3:7-16, 2009; Saunder K O. Front Immunol 10:1296-1316, 2019). Additionally, Fc domains may be modified to enhance neonatal Fc receptor (FcRN) binding that in turn may improve their serum half-life (Dall'Acqua W F et al. J Biol Chem 281:23514-23524, 2006. Moreover, these modifications can be engineered into various antibody formats, such as bispecific (BSP) antibodies and antibody drug conjugates (ADC) to improve their therapeutic activities.

### Example 1—Screening for Proteins that Suppress Antibody Humoral Immune-Effector Function

Recent studies have found that a subset of tumor-produced proteins elicit suppression of antibody-mediated killing effects via ADCC, ADCP and/or CDC (Kline J B, et al. J Clin Oncol 5:15, 2018; Kline J B et al. Eur J Immunol. 48:1872-1882, 2018; Grasso L et al. Oncol Letters 23:2, 2022). In addition, it has been shown that antibodies bound to such proteins are negatively affected in antibody drug conjugate formats (Nicolaides N C. et al. PloS ONE DOI.org/10.1371/journal.pone.0285161, 2023). The immunosuppressive activity appears to occur by direct binding to particular regions of affected antibodies. In attempts to identify additional humoral immunosuppressive proteins, candidate proteins that have been reported by others to be produced by various types of tumor and associated with poor prognosis have been tested using molecular and biological assays to determine their potential as antibody immunosuppressors. The ICAM-1 protein has been reported by several groups to be associated with various cancers and associated with poor prognosis. To evaluate its effect on antibody immune-effector activity, soluble ICAM-1 (sICAM-1) was evaluated for antibody binding. The screening assay employed a 96-well plate ELISA, whereby wells were coated with SICAM-1 protein (Sino Biologicals) protein (SEQ ID NO: 2) and probed with biotinylated human antibodies and measured for binding. As shown in FIG. 1A, wells containing sICAM-1 were bound by all IgG1 antibodies tested in contrast to wells coated with the human serum albumin (HSA) protein that was used as a negative control. Next, ICAM-1/IgG1 binding was tested using immobilized IgG1 or IgM type antibodies and a biotinylated sICAM-1 or HSA. As shown in FIG. 1B, wells containing IgG1-type antibodies and probed with sICAM-1 showed robust binding in contrast to those containing control proteins (IgM, HSA), demonstrating the ability of sICAM-1 to directly bind to IgG1-type antibodies.

The consequences of a protein binding to an IgG1-type antibody may or may not have an impact on its ability to suppress its immune-effector activities. To test the impact of SICAM-1 binding to IgG1-type antibody on immune-effector activity, biological assays monitoring ADCC activity were employed. Reports have shown that membrane-bound ICAM-1 plays a role in enhancing the ADCC of natural killer (NK) cells via LFA binding, and blocking this interaction reduces its ADCC activity on target cells (Cooley S et al. Exp Hematol 27:1533-1541, 1999; Sanchez-Martinez D et al. Theranostics 8:3856-3869, 2018). In order to avoid potential complications of the ICAM-1/LFA pathway on interpreting the effect of sICAM-1 IgG1 mediated ADCC, we employed the Jurkat-CD16a-Luciferase (Jurkat-CD16a) system (Promega) that monitors CD16a activation, a prerequisite for ADCC activity by effector cells. For this assay, CD20-positive Daudi cells were used as target cells and the anti-CD20 rituximab (SEQ ID NO: 4) was used as targeting IgG1 antibody. Briefly, 2×10⁴ target cells were seeded overnight in black opaque 96 well plates in triplicate in R1 assay buffer (RPMI+L-glutamine+1% ultra-low Ig serum) (Gibco). The following day, 1×10⁵ Jurkat-CD16a effector cells were added to wells in R1 assay buffer for an effector target ratio of 5:1 along with 2.5 μg/mL of rituximab and varying amounts of sICAM-1 (ranging from 0 to 10 μg/mL) and plates were incubated for 16 hours at 37° C. in 5% CO₂. After incubation, microwell plates were equilibrated at room temperature for 30 minutes and Jurkat-CD16a activation was measured using the BIO-GLO luciferase reagent following the manufacturer's protocol (Promega). CD16a activation was quantitated using a Varioskan LUX plate reader (ThermoFisher). As shown in FIG. 2A, SICAM-1 had a significant dose-response effect (P<0.003) on suppressing rituximab CD16a activation against Daudi cells. To confirm the ability of sICAM-1 to have effects on other IgG1-type antibodies, similar Jurkat-CD16a activation analyses were conducted using trastuzumab, pertuzumab, cetuximab and rituximab (used a repeat control) at 1 μg/mL in the presence of 10 μg/mL sICAM-1. For trastuzumab and pertuzumab, the HER2-expressing human SK-BR-3 breast cancer cell line was used as target cell. For cetuximab, the EGFR-expressing A431 epidermoid carcinoma cell line was used as target cell. As shown in FIG. 2B, sICAM-1 was able to suppress the CD16a activation of all four antibodies (P<0.008). SICAM-1 toxicity assays on effector and target cells found the cells to tolerate well coincubation at 10 μg/mL concentrations, ruling out any potential artifacts from the above assays.

To demonstrate the effect of ICAM-1 on suppressing IgG1 immune-effector activity, isogenic ICAM-1 knock-down cells were generated using the ICAM-1 expressing HCT116 cell line. Briefly, these cells were generated by seeding 7.5×10⁵ cells in 3 mLs of complete RPMI (RPMI-1640 plus 7.5% fetal bovine serum, 1% L-glutamine) in 6 well plates overnight at 37° C. in 5% CO2. The next day, Lipofectamine 3000 reagent (Sigma) was used to transfect shRNA constructs (SEQ ID NO: 10-13) targeting the human ICAM-1 sequence (Origene TG312270) as per manufacturer's instructions. The 4 independent sequences as well as a scrambled sequence negative control were employed. After transfection, cells were selected for stable construct integration using puromycin selection that was included in the expression construct. After selection, cultures were single-cell cloned in 96-well plates and protein lysate samples screened for the loss of ICAM-1 expression by western blot with a 1:1000 dilution of anti-hICAM-1 rabbit polyclonal antibody (Sino Biologicals) and methods as previously described. Knockdown clones were expanded for banking and tested for decreased IgG1 immune-effector assays to confirm the immunosuppressive effect of sICAM-1 and membrane bound ICAM-1 as compared to isogenic parental cells.

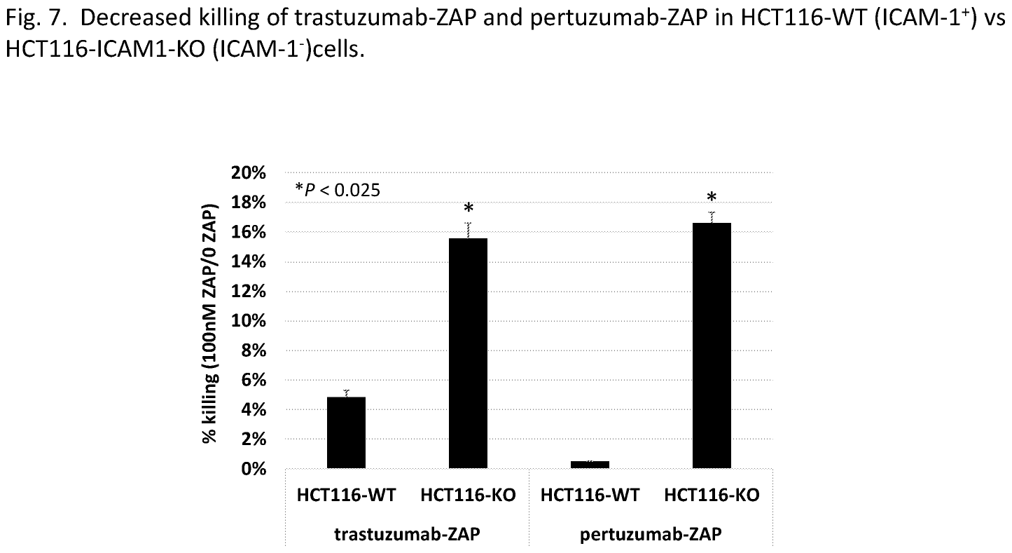

In addition, cells were used to monitor the inhibitory effect of membrane-bound ICAM-1 on IgG1 antibody drug conjugate (ADC) activity due to the potential suppression of internalization via the physical interaction of ICAM-1 with the IgG1 Fc domain. Previous studies of antibodies in ADC format have shown that antibody interacting proteins such as MUC16/CA125 can perturb internalization of ADCs and reduce their target cell toxicity (Nicolaides N C et al. (PLOS ONE May 17; 18 (5):e0285161, 2023). To determine the impact that ICAM-1 may have on antibodies in ADC format, we employed the ZAP streptavidin-saporin antibody-linked system following the manufacturer's (Advanced Targeting Systems) protocol. ZAP links the saporin toxin to biotinylated antibodies and once the antibody-saporin complex is internalized into antigen expressing target cells, the saporin is liberated and becomes to toxic due to ribosome inhibition (Polito, L, et al. Toxins 5:1698-1722, 2013). Briefly, both trastuzumab and pertuzumab antibodies were biotinylated using the EZ-link biotinylation agent following the manufacturer's protocol (ThermoScientific). Next, 100 nM of biotinylated antibody and 100 nM of streptavidin-saporin were added to RPMI 7.5% FBS growth media, and 100 μLs were added per well to HCT116 wildtype and HCT-116-ICAM-1 knockdown cells plated at 5,000 cell/well in clear 96-well microplates. Wells with 100 nM ZAP only and no ZAP antibody were used as negative controls. Plates were incubated for 5 days at 37° C. in 5% CO₂, then analyzed for trastuzumab-ZAP and pertuzumab-ZAP killing using crystal violet blue and quantified using a Varioskan plate reader at 576 nm. As shown in FIG. 7, both trastuzumab-ZAP and pertuzumab-ZAP had significant killing of HCT116-ICAM-1 knockdown cells (HCT116-ICAM1-KO) as compared to HCT116 wildtype (HCT116-WT) cells (P<0.025). The wild-type and knock-out lines expressed similar amounts of HER2 receptor. All experiments were done in triplicate and statistical analysis were done using the Student's T-test. These data support the finding that ICAM-1 binding to antibody in ADC format may perturb their internalization activity and target cell toxicity. The generation of antibodies lacking or with altered ICAM-1 binding (IC1) domain is a useful invention for obtaining optimized ADCs. Alternatively, patient tumors may be screened for ICAM-1 membrane expression and those lacking ICAM-1 expression may be more suitable for ADC-based therapy.

The effect of tumor immunosuppressive proteins on antibody immune-effector activity has previously been shown to be a result of reduced CD16a and/or C1q binding to the Fc IgG domain (Kline J B, et al. J Clin Oncol 5:15, 2018; Kline J B et al. Eur J Immunol. 48:1872-1882, 2018). To evaluate the ability of sICAM-1 to inhibit either of these proteins to bind to the IgG1 Fc domain, ELISA assays were carried out as previously described (Kline J B, et al. Oncotarget 8:52045-52060, 2017; Kline J B et al. Eur J Immunol. 48:1872-1882, 2018). Briefly, 96-well plates were coated with 1 μg/mL of pertuzumab or HSA (used as a negative control) overnight in 0.05M carbonate buffer at 4° C. The next day, wells were washed with phosphate buffer pH 7.2 (PB) and blocked with 5% BSA in PB for 1 hour, then washed three times in PB. Next, wells were probed with 2.5 μg/mL of biotinylated soluble human CD16a (Sino Biologicals) or 1 μg/mL of a biotinylated C1q (Sigma) and secondarily probed with streptavidin-horseradish peroxidase (HRP) for 1 hr at room temp. Wells were then washed 3 times in PB and assayed for binding using TMB colorimetric substrate. Reactions were stopped with 0.1N H₂SO₄ and plates were quantified via 450 nm optical density (A) on a Varioskan plate reader. As shown in FIGS. 3A and 3B, sICAM-1 significantly suppressed the binding of CD16a Fc receptor (P=0.0039) and C1q (P=0.0033), respectively. Similar results were observed for other antibodies tested using the same methods. These data confirm the inhibitory effects of ICAM-1 on antibody humoral immune-effector activity upon physical binding.

### Example 2—Identifying Critical Residues Required for ICAM-1 Binding and Generation of ICAM-1-Refractory IgG1-Type Antibodies

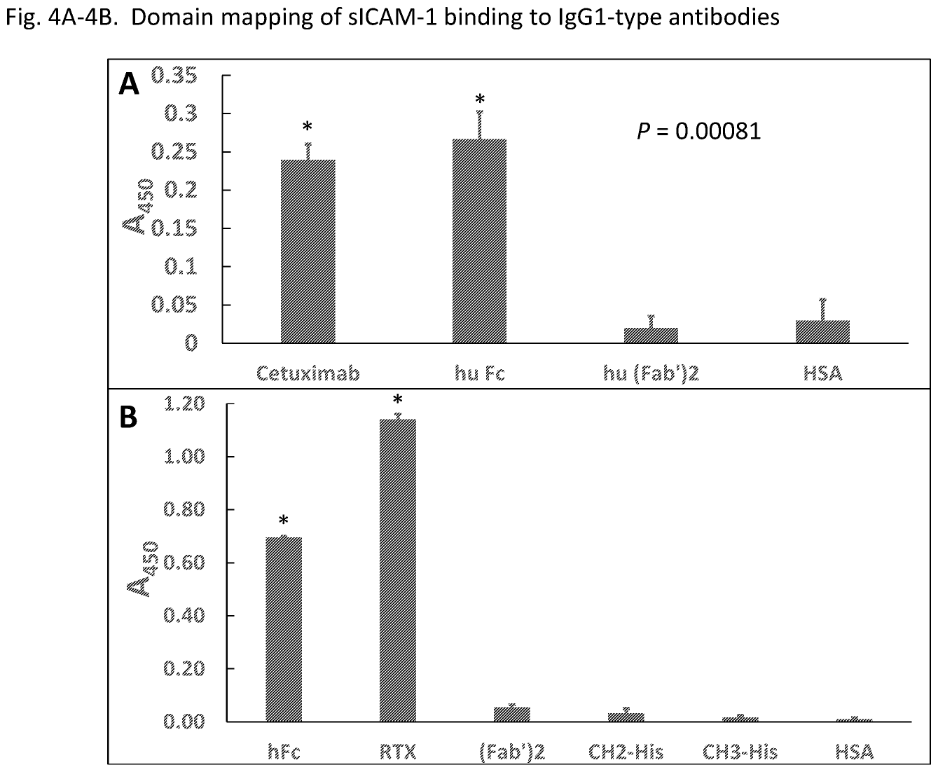

A strategy to identify the binding site or domain to which ICAM-1 bound was employed using IgG1 fragmentation followed by site-directed mutagenesis. First, fragment domain analysis was performed. Human IgG1 antibody was digested by papain to yield F(ab′)2 and Fc fragments that were purified via protein A. The F(ab′) 2 was isolated from the protein A flow through while Fc fragments were captured by the protein A and eluted as previously described (Grasso L et al. Oncol Letters 23:2, 2022). Isolated fragments were quantified via Nanodrop (ThermoFisher) and equal amounts were used in ELISA format to localize sICAM-1 binding. Briefly, plates were coated with 100 μL of 2.5 μg/mL of full length IgG1 antibody or fragments for 1 hour at room temperature in 0.05 M carbonate buffer. Plates were washed in phosphate buffered saline (PBS) plus 0.1% tween-20 (PBS-T) and blocked with 200 uLs of PBS plus 1% BSA. Wells were then washed with PBS-T and probed with 0.5 μg/mL biotinylated sICAM-1 in PBS plus 1% BSA in triplicate on a shaking platform for 1 hour at room temperature. Plates were then washed three times with PBS-T and secondarily probed with 333 ng/ml of streptavidin-HRP in PBS-1% plus BSA for 1 hour at room temperature on a shaking platform. Plates were then washed three times with PBS-T, and quantified using TMB colorimetric substrate (Pierce). Reactions were stopped using 0.1N H₂SO₄ and A450 quantitated using a Varioskan plate reader. As shown in FIG. 4A-4B, SICAM-1 binds to the Fc domain (P=0.00081, as compared to HSA).

To delineate further where within the Fc domain sICAM-1 bound, deletion mutagenesis and ELISA competition assays were performed. A cDNA fusion construct comprising an Ig leader sequence, GST protein, and an IgG1 fragment consisting of the region hinge through CH3 domain of IgG1 followed by a C-terminal FlagTag was synthesized (Genscript) and subcloned into a CMV promoter driven eukaryotic expression plasmid. For deletion mutagenesis, 5′ primers encoding HindIII and Kozak sequences, and a 3′ primer encoding EcoRI and FlagTag were used to amplify fragments from the full-length Fc construct and each fragment was isolated and subcloned into the expression plasmid. For site-directed mutagenesis, overlapping primers encoding the codon changes were used to PCR amplify 5′ and 3′ regions, purified, then used in a second round PCR using flanking primers and fragments were cloned into the expression plasmid. All constructs were DNA sequenced to confirm accuracy. Culture supernatants were analyzed via western blot using the anti-FLAG antibody to ensure proper molecular weight and quantification. Constructs were stably transfected into 293F cells and supernatants were harvested to test the mutant protein's ability to complete with WT IgG1 via ELISA assay.

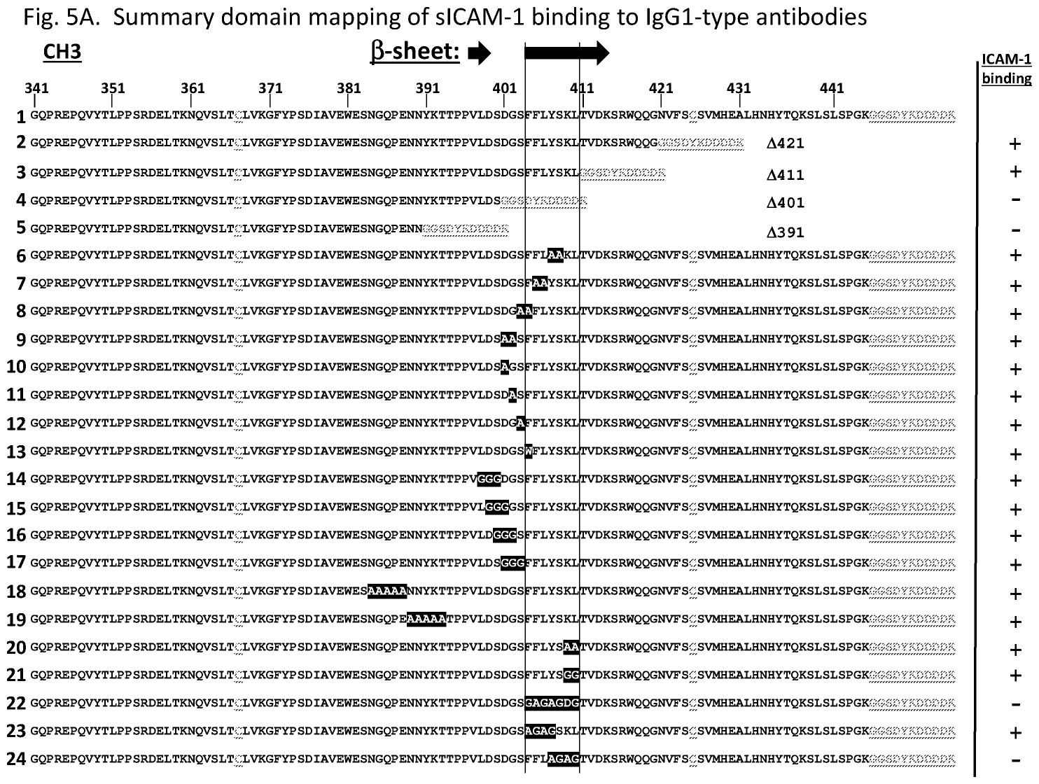

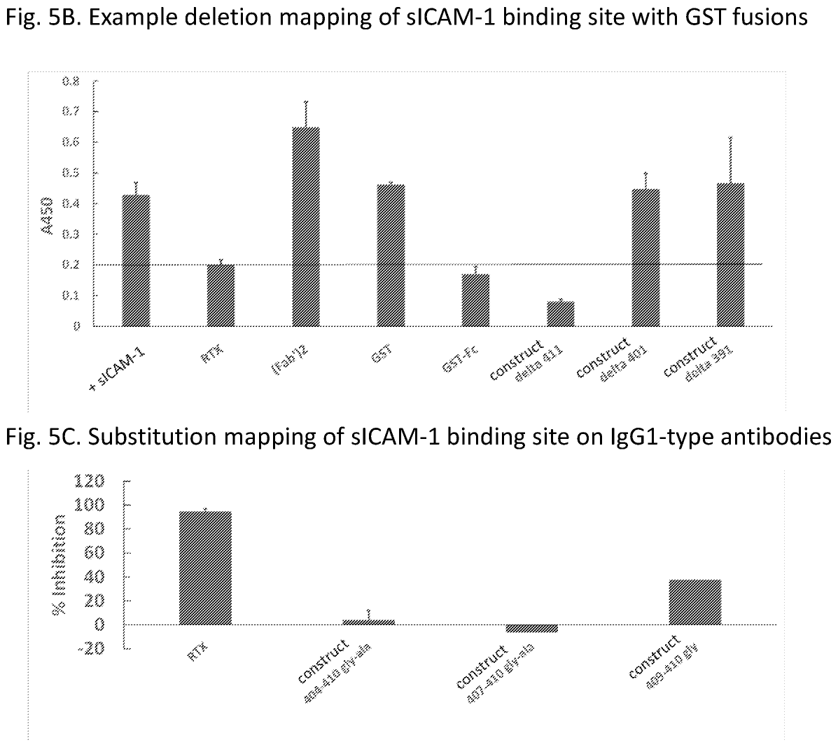

For ELISA competition assays, 96 well plates were coated with 2.5 μg/mL rituximab in 0.05 M carbonate buffer for 1 hour at room temperature. Plates were blocked as described above. After blocking, supernatants containing similar amounts of the various expressed fragments were added in triplicate in conjunction with 0.5 μg/mL biotinylated or 1.5 μg/mL His-tagged full length sICAM-1 and probed for 1 hour at room temperature on a shaking platform. Plates were washed three times with PBS-T and secondary probed with 333 ng/mL streptavidin-HRP for biotinylated ICAM-1 or a 1:3,000 dilution of anti-His-HRP (Sino Biologicals) in PBS plus 1% BSA for 1 hour on a shaking platform. Plates were then washed three times with PBS-T, followed by TMB substrate for colorimetric quantitation. Reactions were stopped with 0.1N H₂SO₄ and A450 quantitated using a Varioskan plate reader. Mutants that lose sICAM-1 binding cannot compete with full length sICAM-1 binding to rituximab coated on the plate and are determined to contain critical residues. As summarized in FIG. 5A and demonstrated in FIGS. 5B and 5C, this domain and amino acids are located to the region Kabat 407 to 410. Further analysis using amino acid substitutions within this region confirmed that these sequences were important for ICAM-1 binding as determined by competitive ELISA screens as described above. As shown in FIG. 8, additional amino acid substitution analysis unexpectedly revealed that amino acid residues proximal to these motifs, e.g., Kabat 369-372 and Kabat 374-377 could also negatively impact ICAM-1 binding to IgG1, thus indicating that modifications within the Kabat region 369-410 can be used for creating ICAM-1 refractory antibodies and antibody drug conjugates.

### Example 3—Engineering ICAM-1 Refractory IgG1 Antibodies to Improve Immune-Effector Activity for Maximal Tumor Cell Killing of Naked IgG1-Based Therapies and Antibody Drug Conjugates (ADCs)

Based on the results described in EXAMPLE 2, one can now develop ICAM-1 refractory IgG1-type antibodies to maximize immune-effector activity and potentially antibody drug conjugates (ADCs) by developing IgG1 Fc domains that are refractory to ICAM-1 binding. As described above, the ability to employ isogenic cells where ICAM-1 protein is knocked down or knocked out permits the screening of IgG1 mutations within or in proximity to the IC1-region. Moreover, the ability to generate IgG1 heavy chain cassettes with modifications as depicted in FIG. 6 or any one, two, three, or four amino acid substitutions within SEQ ID NO: 3 (or in proximity to it, e.g., Kabat 369-372 or Kabat 374-377) permit the development of enhanced IgG1 molecules with improved immune-effector activities, as well as ADCs that are not impeded by ICAM-1 binding and reduced internalization rates, parameters that are critical for maximal ADC target cell killing.

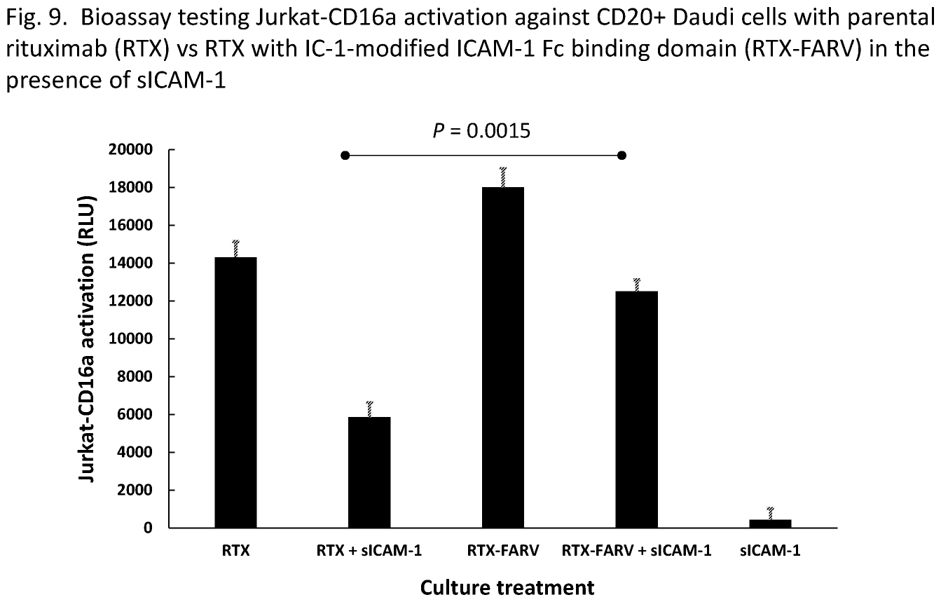

The use of IC1-modified IgG1s are extremely useful in the presence of ICAM-1 positive cancers. These cancers include but are not limited to lymphoma, multiple myeloma, breast cancer, gastric cancer, head and neck cancer and colorectal cancer. Patients can be first screened to determine ICAM-1 status and patients expressing elevated ICAM-1 that is 5% above the normal range as determined by immunohistochemistry or via serum ELISA that can monitor sICAM-1 can benefit from an IC1-modified antibody. These include but are not limited to IC1-modified rituximab (SEQ ID NO: 4 and 5), daratumumab (SEQ ID NO: 9), trastuzumab (SEQ ID NO: 6), pertuzumab (SEQ ID NO: 7) and cetuximab (SEQ ID NO: 8). As shown in FIG. 9, an IC1-modified rituximab (contains four modified amino acids within Kabat 407-410, referred to as RTX-FARV) showed significantly enhanced ADCC activity in the presence of sICAM-1 using the Jurkat-CD16a reporter cell line and Daudi target cell assay as described in EXAMPLE 1, in comparison to parental rituximab (RTX), P=0.0015. These results support the use of improved antibodies for treating diseases that involve overexpression of the immunosuppressive ICAM-1 protein.

All references cited in this document are expressly incorporated herein.

AMINO ACID SEQUENCES
(mature membrane bound human ICAM-1/CD54)
SEQ ID NO: 1
QTSVSPSKVILPRGGSVLVTCSTSCDQPKLLGIETPLPKKELLLPGNNRKVYELSNVQEDSQ
PMCYSNCPDGQSTAKTFLTVYWTPERVELAPLPSWQPVGKNLTLRCQVEGGAPRANLTVVLL
RGEKELKREPAVGEPAEVTTTVLVRRDHHGANFSCRTELDLRPQGLELFENTSAPYQLQTFV
LPATPPQLVSPRVLEVDTQGTVVCSLDGLFPVSEAQVHLALGDQRLNPTVTYGNDSFSAKAS
VSVTAEDEGTQRLTCAVILGNQSQETLQTVTIYSFPAPNVILTKPEVSEGTEVTVKCEAHPR
AKVTLNGVPAQPLGPRAQLLLKATPEDNGRSFSCSATLEVAGQLIHKNQTRELRVLYGPRLD
ERDCPGNWTWPENSQQTPMCQAWGNPLPELKCLKDGTFPLPIGESVTVTRDLEGTYLCRARS
TQGEVTRKVTVNVLSPRYEIVIITVVAAAVIMGTAGLSTYLYNRQRKIKKYRLQQAQKGTPM
KPNTQATPP
(soluble human ICAM-1/CD54)
SEQ ID NO: 2
QTSVSPSKVILPRGGSVLVICSTSCDQPKLLGIETPLPKKELLLPGNNRKVYELSNVQEDSQ
PMCYSNCPDGQSTAKTFLTVYWTPERVELAPLPSWQPVGKNLTLRCQVEGGAPRANLTVVLL
RGEKELKREPAVGEPAEVTTTVLVRRDHHGANFSCRTELDLRPQGLELFENTSAPYQLQTFV
LPATPPQLVSPRVLEVDTQGTVVCSLDGLFPVSEAQVHLALGDQRLNPTVTYGNDSFSAKAS
VSVITAEDEGTQRLICAVILGNQSQETLQTVTIYSFPAPNVILIKPEVSEGTEVTVKCEAHPR
AKVTLNGVPAQPLGPRAQLLLKATPEDNGRSFSCSATLEVAGQLIHKNQTRELRVLYGPRLD
ERDCPGNWTWPENSQQTPMCQAWGNPLPELKCLKDGTFPLPIGESVTVTRDLEGTYLCRARS
TQGEVTRKVTVNVLSPRYE
407-410
SEQ ID NO: 3
YSKL
(rituximab heavy chain with an IC1-region bold-underlined)
SEQ ID NO: 4
QVQLQQPGAELVKPGASVKMSCKASGYTFTSYNMHWVKQTPGRGLEWIGAIYPGNGDTSYNQ
KFKGKATLTADKSSSTAYMQLSSLTSEDSAVYYCARSTYYGGDWYFNVWGAGTTVTVSAAST
KGPSVFPLAPSSKSTSGGTAALGCLVKDYFPEPVTVSWNSGALISGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKENWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
(rituximab-N109D with the heavy chain with an IC1-region and
CA125-refractory residue bold-underlined)
SEQ ID NO: 5
QVQLQQPGAELVKPGASVKMSCKASGYTFTSYNMHWVKQTPGRGLEWIGAIYPGNGDTSYNQ
KFKGKATLTADKSSSTAYMQLSSLISEDSAVYYCARSTYYGGDWYFDVWGAGTTVTVSAAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFP
PKPKDTLMISRTPEVTCVVVDVSHEDPEVKENWYVDGVEVHNAKTKPREEQYNSTYRVVSVL
TVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHE
ALHNHYTQKSLSLSPGK
(trastuzumab heavy chain with an IC1-region bold-underlined )
SEQ ID NO: 6
EVQLVESGGGLVQPGGSLRLSCAASGFNIKDTYIHWVRQAPGKGLEWVARIYPINGYTRY
ADSVKGRFTISADTSKNTAYLQMNSLRAEDTAVYYCSRWGGDGFYAMDYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKENWYVDGVEVHNAKTKPREEQYN
STYRVVSVLIVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRW
QQGNVFSCSVMHEALHNHYTQKSLSLSPGK
(pertuzumab heavy chain with an IC1-region bold-underlined)
SEQ ID NO: 7
EVQLVESGGGLVQPGGSLRLSCAASGFTFTDYTMDWVRQAPGKGLEWVADVNPNSGGSIY
NQRFKGRFTLSVDRSKNTLYLQMNSLRAEDTAVYYCARNLGPSFYFDYWGQGTLVTVSSA
STKGPSVFPLAPSSKSTSGGTAALGCLVKDYFPEPVTVSWNSGALISGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKENWYVDGVEVHNAKTKPREEQYNS
TYRVVSVLIVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPGK
(cetuximab heavy chain with an IC1-region bold-underlined)
SEQ ID NO: 8
QVQLKQSGPGLVQPSQSLSITCTVSGFSLINYGVHWVRQSPGKGLEWLGVIWSGGNTDYN
TPFTSRLSINKDNSKSQVFFKMNSLQSNDTAIYYCARALTYYDYEFAYWGQGTLVTVSAA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQ
QGNVFSCSVMHEALHNHYTQKSLSLSPGK
(daratumumab heavy chain with an IC1-region bold-underlined)
SEQ ID NO: 9
EVQLLESGGGLVQPGGSLRLSCAVSGFTENSFAMSWVRQAPGKGLEWVSAISGSGGGTYY
ADSVKGRFTISRDNSKNTLYLQMNSLRAEDTAVYFCAKDKILWEGEPVFDYWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVHNAKTKPREEQ
YNSTYRVVSVLIVLHQDWLNGKEYKCKVSNKALPAPIEKTISKAKGQPREPQVYTIPPSR
EEMTKNQVSLTCLVKGFYPSDIAVEWESNGQPENNYKITPPVLDSDGSFFLYSKLTVDKS
RWQQGNVFSCSVMHEALHNHYTQKSLSLSPGK
SEQ ID NO: 10, 11, 12, 13 (ICAM-1 shRNAs)
shRNA 312270A 1
GGATCGCACTGTGGTAGCAGCCGCAGTCATAATGGTCAAGAGCCATTATGACTGCGGCTG
CTACCACAGTGTTTTTTGAAGCTT
shRNA 312270B 2
GGATCGTACCTCTATAACCGCCAGQGGAAGATCAATCAAGAGTTGATCTTCCGCTGGGGG
TTATAGAGGTATTTTTTGAAGCTT
shRNA 312270C 3
GGATCGACCTTCCTCACCGTGTACTGGACTCCAGATCAAGAGTCTGGAGTCCAGTACACG
GTGAGGAAGGTTTTTTTGAAGCTT
shRNA 312270D 4
GGATCGATTTCTCGTGCCGCACTGAACTGGACCTGTCAAGAGCAGGTCCAGTTCAGTGCG
GCACGAGAAATTTTTTTGAAGCTT

1. An immunosuppression-refractory human IgG1 antibody comprising from one to four amino acid substitutions relative to an immunosuppression-susceptible human IgG1 antibody without the one to four amino acid substitutions, wherein immunosuppression of the immunosuppression-susceptible human IgG1 is caused by soluble or membrane bound ICAM-1/CD54.

2. The immunosuppression-refractory human IgG1 antibody of claim 1 which is a full-length antibody.

3. The immunosuppression-refractory human IgG1 antibody of claim 1 which is an antibody drug conjugate.

4. The immunosuppression-refractory human IgG1 antibody of claim 1 wherein the ICAM-1/CD54 is membrane-bound ICAM-1/CD54 as shown in SEQ ID NO: 1.

5. The immunosuppression-refractory human IgG1 antibody of claim 1 wherein the ICAM-1/CD54 is soluble ICAM-1/CD54 as shown in SEQ ID NO: 2.

6. The immunosuppression-refractory human IgG1 antibody of claim 1 wherein the one to four amino acid substitutions are within Kabat residues 367-425.

7. The immunosuppression-refractory human IgG1 antibody of claim 1 wherein the one to four amino acid substitutions are within Kabat residues 369-410.

8. The immunosuppression-refractory human IgG1 antibody of claim 6 where the one to four amino acid substitutions are within the motif YSKL (SEQ ID NO: 3) which is located within a heavy chain of the immunosuppression-refractory human IgG1 antibody.

9. The immunosuppression refractory human IgG1 antibody of claim 6 which comprises an IgG1 light chain and a IgG1 heavy chain.

10. The immunosuppression refractory human IgG1 antibody of claim 1 which is conjugated to a drug.

11. A polynucleotide encoding the immunosuppression-refractory human IgG1 antibody of claim 1.

12. A nucleic acid vector encoding the immunosuppression-refractory human IgG1 antibody of claim 1.

13. A stable cell line comprising the nucleic acid vector of claim 12 wherein the stable cell line expresses the immunosuppression-refractory human IgG1 antibody.

14. A method of treating a patient with a disease, wherein the patient expresses an elevated level of ICAM-1/CD54 compared to a population of healthy humans, the method comprising:
administering to the patient the immunosuppression-refractory human IgG1 antibody of claim 1.

15. The method of claim 14 wherein the disease is cancer.

16. The method of claim 14 wherein the disease is an inflammatory disease.

17. The method of claim 15 wherein the cancer is selected from the group consisting of Hodgkin's Lymphoma, Non-Hodgkin's Lymphoma, Follicular Lymphoma, Large Cell Lymphoma, Diffuse Large B-cell Lymphoma, Chronic Lymphocytic Leukemia, Multiple Myeloma, Non-small Lung Carcinoma, Breast Carcinoma, Colorectal Carcinoma, Gastric Carcinoma, and Head and Neck Carcinoma.

18-27. (canceled)

28. A method of screening candidate antibodies to identify those that are refractory to ICAM-1/CD54 immunosuppression, comprising:
(a) contacting a first human cancer cell line expressing an antigen with a candidate IgG1-type antibody which specifically binds to the antigen, wherein the contacting is performed in the presence of ICAM-1/CD54 (SEQ ID NO: 1);
(b) contacting a second human cancer cell line expressing the antigen with the candidate IgG1-type antibody which specifically binds to the antigen, wherein the contacting is performed in the absence of ICAM-1/CD54 (SEQ ID NO: 1); and (c) determining antibody dependent cellular cytotoxicity (ADCC) or complement dependent cytotoxicity (CDC) of the first and second human cancer cell lines stimulated by the candidate antibody in steps (a) and (b).

29-35. (canceled)

36. A method to identify tumor-bearing patients who are good candidates for treatment with an antibody-drug conjugate, comprising:
testing a tumor from each of a plurality of patients, and determining whether the tumor from each of the plurality of patients expresses ICAM-1;
wherein if the tumor does not express ICAM-1, then recommending treatment of the patient with an IgG1 antibody-drug conjugate, wherein the IgG1 antibody of the antibody-drug conjugate is immunosuppression-susceptible; and
wherein if the tumor expresses ICAM-1, then recommending that the patient not be treated with an IgG1 antibody-drug conjugate, wherein the IgG1 antibody of the antibody-drug conjugate is immunosuppression-susceptible; and
optionally treating at least one patient that does not express ICAM-1 with an IgG1 antibody-drug conjugate, wherein the IgG1 antibody of the antibody-drug conjugate is immunosuppression-susceptible.


SEQUENCE LISTING (13 sequences)

SEQ ID NO: 1 - protein, 505 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 2 - protein, 453 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYE

SEQ ID NO: 3 - protein, 4 aa, Homo sapiens
YSKL

SEQ ID NO: 4 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 449 aa, Homo sapiens
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 452 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 84 aa, Homo sapiens
GGATCGCACTGTGGTAGCAGCCGCAGTCATAATGGTCAAGAGCCATTATGACTGCGGCTGCTACCACAGTGTTTTTTGAAGCTT

SEQ ID NO: 11 - protein, 84 aa, Homo sapiens
GGATCGTACCTCTATAACCGCCAGCGGAAGATCAATCAAGAGTTGATCTTCCGCTGGCGGTTATAGAGGTATTTTTTGAAGCTT

SEQ ID NO: 12 - protein, 84 aa, Homo sapiens
GGATCGACCTTCCTCACCGTGTACTGGACTCCAGATCAAGAGTCTGGAGTCCAGTACACGGTGAGGAAGGTTTTTTTGAAGCTT

SEQ ID NO: 13 - protein, 84 aa, Homo sapiens
GGATCGATTTCTCGTGCCGCACTGAACTGGACCTGTCAAGAGCAGGTCCAGTTCAGTGCGGCACGAGAAATTTTTTTGAAGCCT